(19) United States
(12) Patent Application Publication
(10) Pub. No.: US 2024/0169714 A1
(43) Pub. Date: May 23, 2024

(54) Asymmetric Multi-Modal Machine Learning System and Method using Clinical Metadata in Electronic Medical Records

(71) Applicant: Georgia Tech Research Corporation, Atlanta, GA (US)

(72) Inventors: Ghassan AlRegib, Atlanta, GA (US); Kiran Kokilepersaud, Atlanta, GA (US); Mohit Prabhushankar, Atlanta, GA (US); Yash-yee Logan, Atlanta, GA (US); Ahmad Mustafa, Atlanta, GA (US)

(21) Appl. No.: 18/514,542

(22) Filed: Nov. 20, 2023

Related U.S. Application Data
(60) Provisional application No. 63/384,316, filed on Nov. 18, 2022.
(60) Provisional application No. 63/426,470, filed on Nov. 18, 2022.

(51) Int. Cl. G06V 10/82; G16H 10/60; G16H 30/20

(52) U.S. Cl. CPC G06V 10/82; G16H 10/60; G16H 30/20

(57) ABSTRACT

An exemplary system and method that facilitate the use of clinical medical data in electronic medical records for training an AI model. In an aspect, the exemplary system and method can be used for asymmetric multi-modal machine learning training, e.g., supervised contrastive learning, on one data set modality (e.g., having clinical labels) to learn useful features in a first model for fine-tuning on another data set (e.g., having biomarker labels). In another aspect, the exemplary system and method can use demographic information in electronic medical records for training an AI model.

### RELATED APPLICATION

This US patent application claims priority to, and the benefit of, U.S. Provisional Patent Application No. 63/384,316, filed Nov. 18, 2022, entitled “Multi-modal, Trustworthy, and Unsupervised Active Learning.” and U.S. Provisional Patent Application No. 63/426,470, filed Nov. 18, 2022, entitled “Asymmetric Multi-modal Data Integration,” which is incorporated by reference herein in its entirety.

### BACKGROUND

Deep learning is the subset of machine learning methods that are based on artificial neural networks with representation learning. Deep learning approaches generally rely on access to a large quantity of labeled data. Labeled data can be used as ground truth during the training operation and are generally provided in a supervised manner by radiologists and other specialists for medical diagnostics or imaging.

The dependence of training of deep learning systems on potentially expensive labels makes them sub-optimal for the various constraints of the medical field.

There is a benefit to improving deep learning systems and their associated training.

### SUMMARY

An exemplary system and method are disclosed that facilitate the use of clinical medical data in electronic medical records for training an AI model. In an aspect, the exemplary system and method can be used for asymmetric multi-modal machine learning training, e.g., supervised contrastive learning, on one data set modality (e.g., having clinical labels) to learn useful features in a first model for fine-tuning on another data set (e.g., having biomarker labels). In another aspect, the exemplary system and method can use demographic information in electronic medical records for training an AI model.

To train conventional deep learning architectures, large quantities of labeled data are necessary. In the medical field and other various engineering disciplines, the dependence is often not generalizable. Often, there are application settings where a prolific amount of data exists for one modality while lacking such amounts of large data on another modality. An example is in the ophthalmic domain, where clinical and demographic labels are readily available while physician-interpreted biomarker labels are not so readily available.

Despite the discrepancy, the modalities do share relationships with one another that are a function of their manifestation within the body. To this end, training with data from one modality can transfer the knowledge learned to the one lacking in data. The exemplary system and method employ a supervised contrastive learning operation on one medical modality (e.g., clinical labels) in order to learn useful features for fine-tuning on another modality (e.g., biomarker labels). The exemplary system and method can facilitate the analysis of AI applications where labels are limited in a candidate training data set. Also, embodiments of the present disclosure make it possible to deploy deep learning operations even when access to domain experts is limited. Examples include medical fields, geology, geophysics, astronomy, and many other fields.

A first study was conducted that investigated the usage of a supervised contrastive loss on clinical data to train a model for biomarker classification. The first study observed that the method performed across different combinations of clinical labels can provide new biomarker labels that can be used for hyperparameter tuning. The study concluded, through extensive experimentation on biomarkers of varying granularity within OCT scans, that the usage of clinical labels is a more effective way to leverage the correlations that exist within unlabeled data over traditional supervised and self-supervised algorithms. The first study shows that there are ways to utilize correlations that exist between measured clinical labels and their associated biomarker structures within images. Additionally, our method is based on practically relevant considerations regarding detecting key indicators of disease as well as challenges associated with labeling images for all the different manifestations of biomarkers that could be present.

A second study was conducted that trained a supervised contrastive loss in order to train an encoder network to learn the distinguishing characteristics of seismic data from a contrastive loss. Training in this manner led to a representation space more consistent with the seismic setting and was shown to out-perform a state-of-the-art self-supervised methodology in a semantic segmentation task.

Multi-modal, Trustworthy, and Unsupervised Active Learning. In another aspect, active learning aims to reduce the time and cost associated with data annotation. However, at the beginning of an active learning workflow, there is not enough visual data. Nevertheless, there is data present in other modalities like clinical labels, demographic information, biomarkers, log data, and data samples, among other examples from a variety of applications.

Another exemplary method and system are disclosed that can employ active learning for visual data using data acquired from electronic health records, including clinical labels, demographic information, biomarkers, and log data, among other examples from a variety of applications. By utilizing the additional clinical labels available in the electronic health records, in addition to the radiologic images, to learn and make disease diagnoses in a medical application or a well productivity assessment in a geophysical application, the exemplary method and system can improve training of an AI/ML model at early stages of a project or application when training data is sparse. Because the additional data may be available in multiple formats, like 1D, 2D, tensors, or 3D, the fusion of these heterogeneous modalities may not be straightforward. While prior methods may use fusion (late or early), it may require even larger networks without enough data.

The second exemplary system and method employ a sampling strategy during training to develop a framework that can expand and generalize to any data modality and application. In a medical diagnosis application, this can be achieved by sampling identity, BCVA, CST, or any other auxiliary data type.

A third study was conducted that validated the exemplary system and method by retaining the performance at the previous round and ensuring that there was minimal regression in model performance. In doing so, the study created a trustworthy and multi-modal algorithm. Specifically, the third study augmented active learning paradigms with EMR data about patient identity.

In an aspect, a method is disclosed for asymmetric training an AI model, the method comprising: receiving a multi-modal dataset including a first image data set and a second image data set, wherein the first image data set includes a meta data label as a medical condition in an electronic medical record of a patient, and wherein the second image data set includes clinical labels (e.g., biomarkers); performing training of an AI model using the first dataset using the meta data labels to adjust first weights in the AI model; and performing contrastive learning of the AI model using the second dataset, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the meta data label.

In another aspect, a method is disclosed for using a asymmetrically train AI model, the method comprising: receiving, by a processor, a image data set acquired from a scanner; determining, by the processor, via a trained AI model, the presence or non-presence of a disease or medical condition, wherein the trained AI model was configured using a multi-modal dataset that includes a first image data set and a second image data set, wherein the first image data set includes a meta data label as a medical condition in an electronic medical record of a patient, and wherein the second image data set includes clinical labels (e.g., biomarkers), wherein the training of the AI model used the meta data labels to adjust first weights in the AI model and used the second dataset in contrastive learning of the AI model, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the meta data label; and outputting, via a graphical user interface or report, the determined presence or non-presence of a disease or medical condition.

In some embodiments, the step of performing the supervised learning of an AI model includes: providing a clinically labeled augmented batch having the meta data label; forward propagating through the AI model; varying a projection network coupled to the AI model; and computing a loss function at the output of the projection network to adjust the AI model.

In some embodiments, the method further includes outputting, via a report or display, a classifier output for diagnosis of the disease or the medical condition.

In some embodiments, the first data set comprises image data from a medical scan.

In some embodiments, the first data set comprises image data from a sensor.

In some embodiments, the first portion of the AI model comprises an autoencoder.

In some embodiments, the second portion of the AI model comprises a linear layer appended to the first portion.

In some embodiments, the second portion of the AI model comprises a semantic segmentation head appended to the first portion.

In some embodiments, the biomarker data includes at least one of: Intraretinal Fluid (IRF), Diabetic Macular Edema (DME), and Intra-Retinal Hyper-Reflective Foci (IRHRF).

In some embodiments, the training operation is configured to: compute a distribution of unique identifiers for subjects throughout an unlabeled pool; and sample for the training operation based on the computed distribution.

In another aspect, a system is disclosed comprising a processor; and a memory having instructions stored thereon, wherein execution of the instructions by the processor causes the processor to: receive a multi-modal dataset including a first image data set and a second image data set, wherein the first image data set includes a meta data label as a medical condition in an electronic medical record of a patient, and wherein the second image data set includes clinical labels (e.g., biomarkers); perform training of an AI model using the first dataset using the meta data labels to adjust first weights in the AI model; and perform contrastive learning of the AI model using the second dataset, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the meta data label.

In some embodiments, the instructions to perform the supervised learning of an AI model includes: instructions to provide a clinically labeled augmented batch having the meta data label; instructions to forward propagating through the AI model; instructions to vary a projection network coupled to the AI model; and instructions to compute a loss function at the output of the projection network to adjust the AI model.

In some embodiments, the system further includes a sensor, wherein the first data set comprises image data acquired from the sensor.

In some embodiments, the first portion of the AI model comprises an autoencoder.

In some embodiments, the second portion of the AI model comprises at least one of (i) a linear layer appended to the first portion or (ii) a semantic segmentation head appended to the first portion.

In some embodiments, the instructions for the training operation includes: instructions to compute a distribution of unique identifier for subject throughout an unlabeled pool; and instructions to sample for the training operation based on the computed distribution.

In another aspect, a non-transitory computer readable medium is disclosed having instructions stored thereon, wherein execution of the instructions by a processor causes the processor to: receive a multi-modal dataset including a first image data set and a second image data set, wherein the first image data set includes a meta data label as a medical condition in an electronic medical record of a patient, and wherein the second image data set includes clinical labels (e.g., biomarkers); perform training of an AI model using the first dataset using the meta data labels to adjust first weights in the AI model; and perform contrastive learning of the AI model using the second dataset, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the meta data label.

In some embodiments, the instructions to perform the supervised learning of an AI model includes: instructions to provide a clinically labeled augmented batch having the meta data label; instructions to forward propagating through the AI model; instructions to vary a projection network coupled to the AI model; and instructions to compute a loss function at the output of the projection network to adjust the AI model.

In some embodiments, the second portion of the AI model comprises at least one of (i) a linear layer appended to the first portion or (ii) a semantic segmentation head appended to the first portion.

In some embodiments, the instructions for the training operation includes: instructions to compute a distribution of unique identifier for subject throughout an unlabeled pool; and instructions to sample for the training operation based on the computed distribution.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 shows an example system configured with analysis module to perform contrastive learning to generate labels for unlabeled medical images to train, via a training module, a classification engine in accordance with an illustrative embodiment.

FIGS. 2A and 2B each shows an example method to perform contrastive learning to generate meta data-based labels for unlabeled medical images to train a classification engine in accordance with an illustrative embodiment.

FIGS. 3A-3K shows experimental results for a first study using contrastive learning to generate meta data-based labels for unlabeled medical images in accordance with an illustrative embodiment. FIG. 3A shows an exemplary method for the supervised contrastive learning in accordance with an illustrative embodiment. FIG. 3B shows an image with biomarkers that were used in the study. FIG. 3C and FIG. 3D show examples of OTC scans used in the study. FIG. 3E shows the distributions within the dataset of the study for the clinical values of BCVA and CST used in the contrastive learning. FIG. 3F shows comparative results of the contrastive training against other algorithms. FIG. 3G shows clinical labels and biomarker labels available in the Prime and TREX-DME data set used in the study. FIG. 3H shows the performance of the contrastive learning as a function of available biomarker training data. FIG. 3I shows the performance of a patient split experiment. FIG. 3J shows performance data based on averaged AUCROC after training the encoder using data from the Prime dataset. FIG. 3K shows performance data of the clinical contrastive method integrated into a semi-supervised framework.

FIGS. 4A-4D shows experimental results for a second study using contrastive learning to generate meta data-based labels for unlabeled seismic images in accordance with an illustrative embodiment. FIG. 4A shows an example of the seismic images. FIG. 4B shows a volume generation process. FIG. 4C shows a flowchart of the contrastive learning. FIG. 4D shows a summary of the comparative results.

FIGS. 5A-5F shows experimental results for a third study using deployable clinical active learning in accordance with an illustrative embodiment. FIG. 5A shows sample scans from each dataset with patients having the same disease. FIG. 5B shows the results of two experimental modalities in the initialization phase based on the availability of data. FIG. 5C shows implementation details for each dataset. FIG. 5D shows the results. FIG. 5E shows the test accuracy vs sample count during training for the OCT on Resnet-18. FIG. 5F shows the test accuracy vs sample count during training for the X-Ray on Densenet-121.

### DETAILED SPECIFICATION

To facilitate an understanding of the principles and features of various embodiments of the present invention, they are explained hereinafter with reference to their implementation in illustrative embodiments.

### Example System

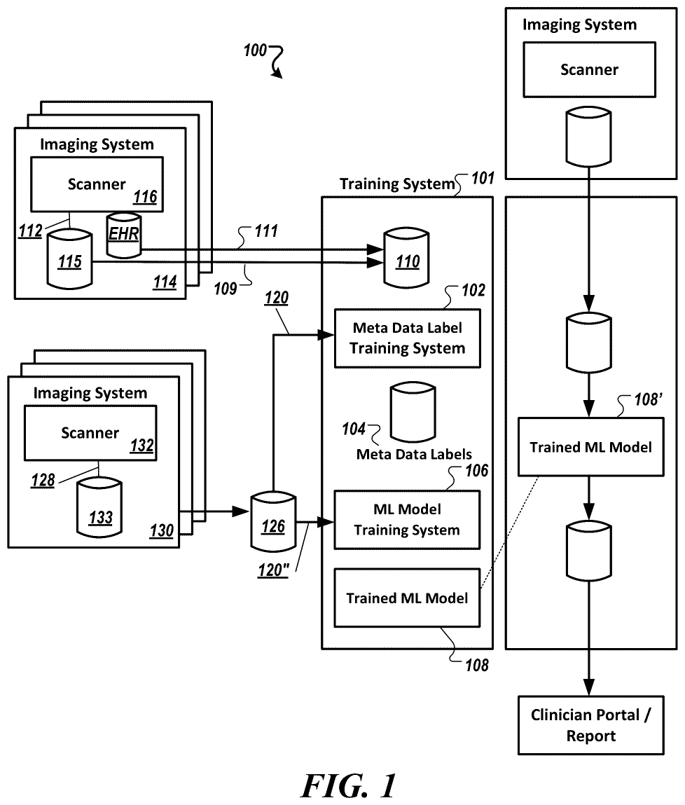

FIG. 1 shows an example system 100 configured with analysis module 102 (shown as “Meta Data Label Training System” 102) to perform contrastive learning to generate labels 104 for unlabeled medical images to train, via a training module 106 (shown as “ML Model Training System” 106), a classification engine 108 (shown as “trained ML model” 108) in accordance with an illustrative embodiment. The classification engine 108 can then be used for diagnostics or treatment of a disease or medical condition.

In the example shown in FIG. 1, the analysis module 102 is configured to receive a training data set 109 and electronic health record data 111 from a data store 110. The data store 110 may be located on an edge device, a server, or cloud infrastructure to receive the scanned medical images 112 from an imaging system 114 comprising a scanner 116. The imaging system 114 can acquire scans for optical coherence tomography, ultrasound, magnetic resonance imaging, and computing tomography, among other modalities described or referenced herein. The scanned data can be stored in a local data store 115 to then be provided as the training data set 109 to the training system 101.

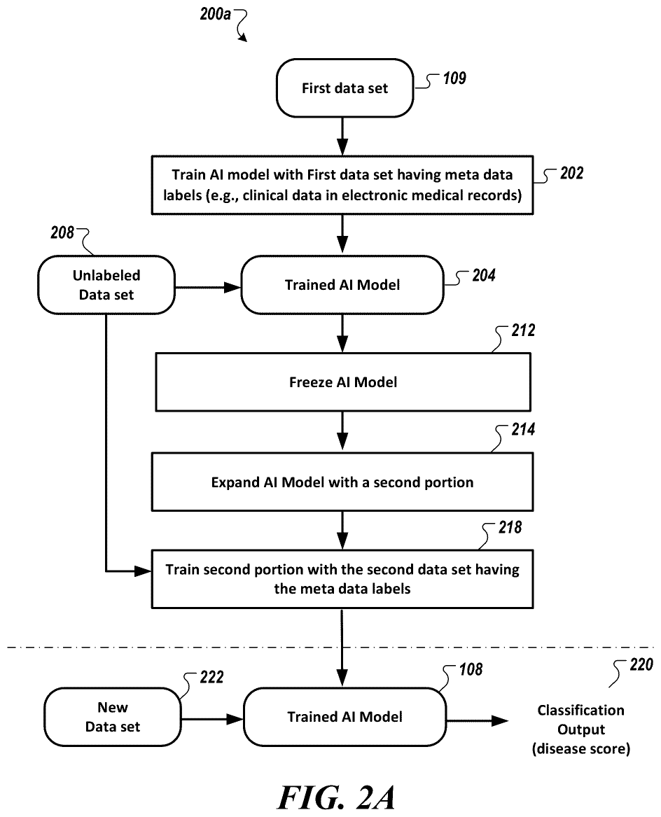

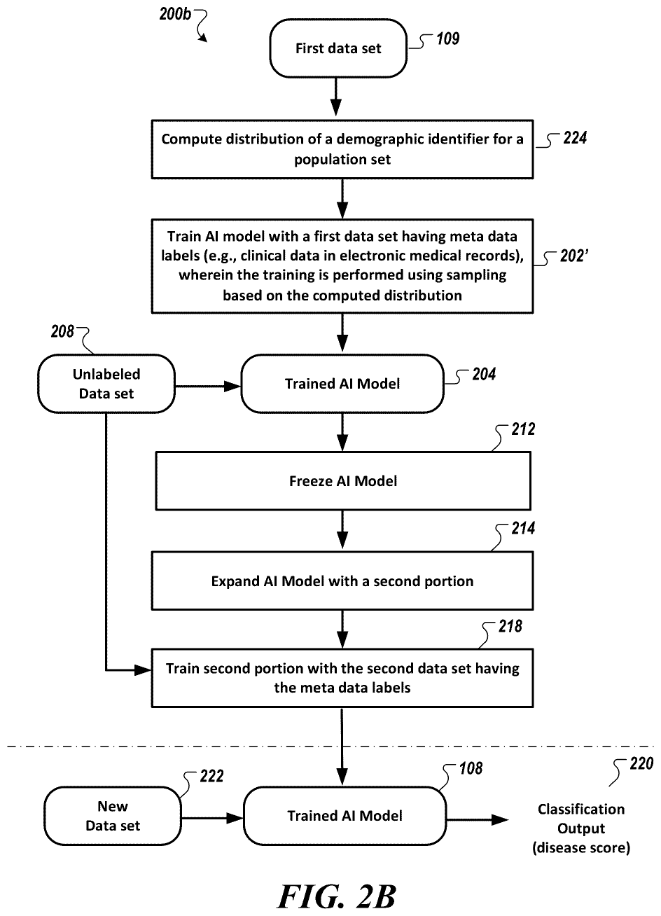

The contrastive-learning training data set 109 is used with an electronic medical record (EMR) data 111 that is first used to train a model (e.g., 204, see FIG. 2A) (e.g., autoencoder or other ML models described herein) of the analysis module 102. The model can then be used to fine-tune a second model with respect to an unlabeled data set 120 (shown as 120″).

In the example shown in FIG. 1, once the meta data clinical labels 104 have been used to select unlabeled medical images, they can be employed in a training operation, via the training module 106, as a second set of training data for the classification engine 108.

In some embodiments, a backbone network f(⋅) is trained with a supervised clinical contrastive loss that uses the clinical label to choose positives and negatives. The weights of the backbone network are frozen and a linear layer can be appended to the output of this network. This layer is fine-tuned using the smaller subset of images containing labels for the modality of information that is much more scarce. It is trained with a cross-entropy loss in order to identify these labels.

The analysis module 102 is configured to receive unlabeled data set 120 from a data store 126. The data store 126 may be located on an edge device, a server, or cloud infrastructure to receive the scanned medical images 128 from an imaging system 130 comprising a scanner 132. The imaging system 130 can acquire scans for optical coherence tomography, ultrasound, magnetic resonance imaging, and computing tomography, among other modalities described or referenced herein. The scanned data can be stored in a local data store 133 to then be provided as the training data set 120 (shown as 120″) to the training system 106 along with the corresponding labels 104.

The training performed at the ML model training system 106 can be performed in a number of different ways. The ML model training system 106 can be employed to use all the generated meta data labels 104, and corresponding data set 120″ for the training, in which the generated labels 104 are employed as ground truth. The resulting classification engine 108 (shown as 108′) can then be used to generate an estimated/predicted meta data label/score for a new data set in a clinical application. In such embodiments, the classification engine 108′ can additionally generate an indication for the presence or non-presence of a disease or medical condition.

Referring still to FIG. 1, the output of the classification engine 108′ can be outputted via a report or display, e.g., for the diagnosis of a disease or a medical condition and/or for the treatment of the disease or a medical condition. Treatment refers to operations of medical instruments that operate on a tissue, e.g., to excise, remove, cut, ablate, or cool a tissue. Treatment can also refer to the introduction (e.g., injection) of a therapeutic agent. In some embodiments, an edge device, server, or cloud infrastructure can employed, e.g., via web services, to curate a clinician or healthcare portal to display the report or information in a graphical user interface.

Biomarker training. The training system 106 can train the metadata labels 104 and associated training dataset 120″, which can be marked with biomarker data. Biomarkers can include any substance, structure, or process that can be measured in the body or its products and influence or predict the incidence of outcome or disease. In the context of Diabetic Retinopathy, biomarkers can include, for example, but not limited to, the presence or degree of Intraretinal Fluid (IRF), Diabetic Macular Edema (DME), Intra-Retinal Hyper-Reflective Foci (IRHRF), atrophy or thinning of retinal layers, disruption of the ellipsoid zone (EZ), disruption of the retinal inner layers (DRIL), intraretinal (IR) hemorrhages, partially attached vitreous face (PAVF), fully attached vitreous face (FAVF), preretinal tissue or hemorrhage, vitreous debris, vitreomacular traction (VMT), diffuse retinal thickening or macular edema (DRT/ME), subretinal fluid (SRF), disruption of the retinal pigment epithelium (RPE), serous pigment epithelial detachment (PED), and subretinal hyperreflective material (SHRM). Additional examples of biomarkers in OCT can be found at [2].

In addition to images, the example system of FIG. 1 can be employed to evaluate other image and sensor data, e.g., optical, temperature, acoustic, sound, strain/stress, etc., as employed in clinical, engineering, and metrology applications. The exemplary system, for example, can be employed for Clinical Disease Detection, Clinical diagnosis analysis, X-ray interpretation, OCT interpretation, Ultrasound Interpretation, Infrastructure Assessment, Structure Integrity assessment, Industrial applications, Manufacturing applications, and Circuit Boards defect detection systems, among others.

### Example Training Operation

FIGS. 2A and 2B each shows an example method 200 (shown as 200a, 200b, respectively) to perform contrastive learning to generate meta data-based labels (e.g., 104) for unlabeled medical images to train a classification engine (e.g., 108) in accordance with an illustrative embodiment. Method (200a, 200b) includes in a contrastive learning operation, training (202) a first ML model 204 via a first data set 109 that includes meta data labels in the form of readily available clinical data in electronic medical records. In the training, the first weights of the AI model are adjusted.

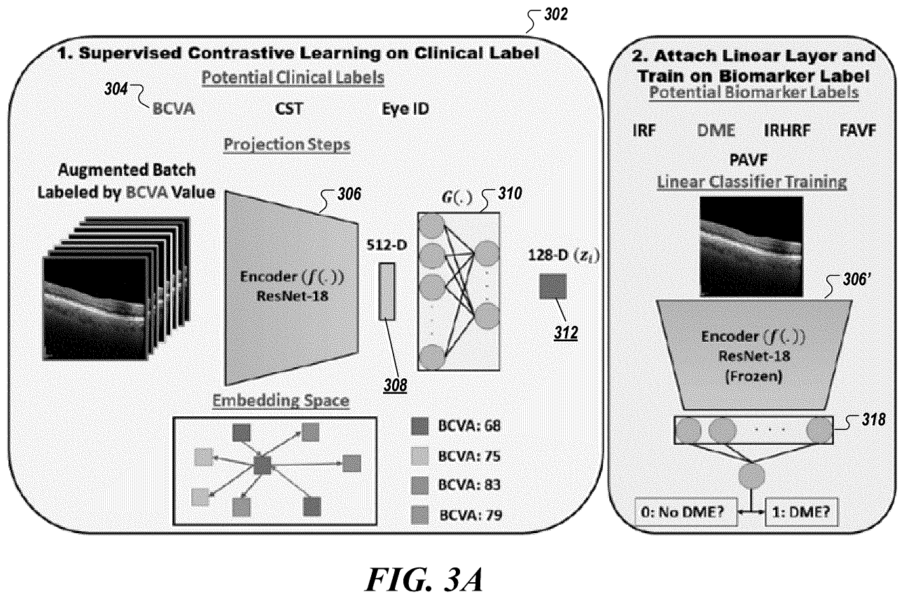

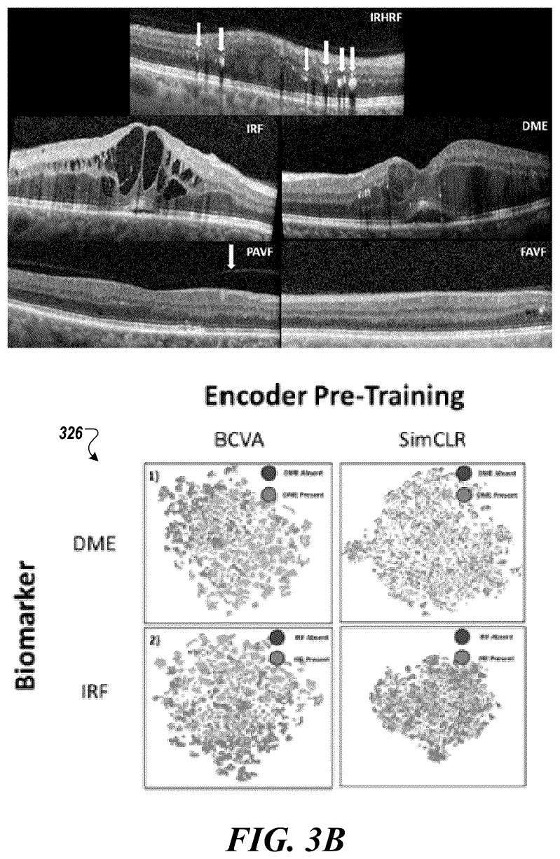

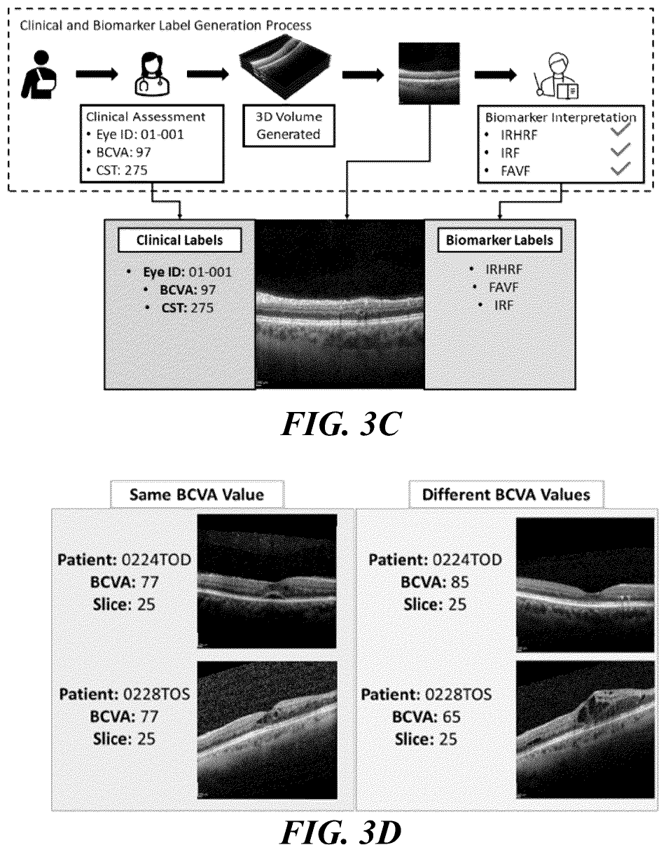

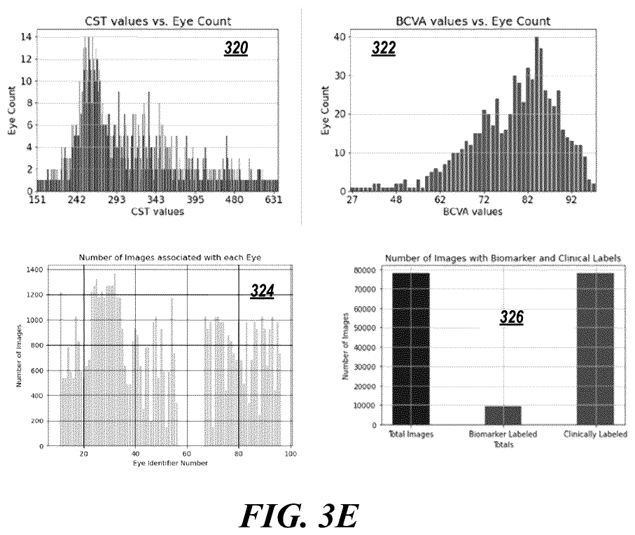

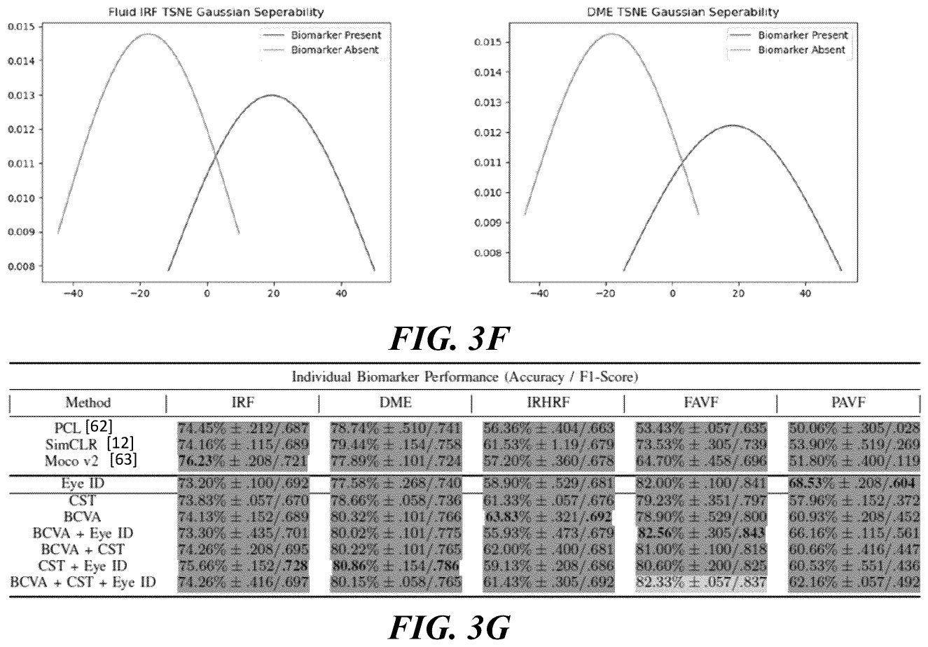

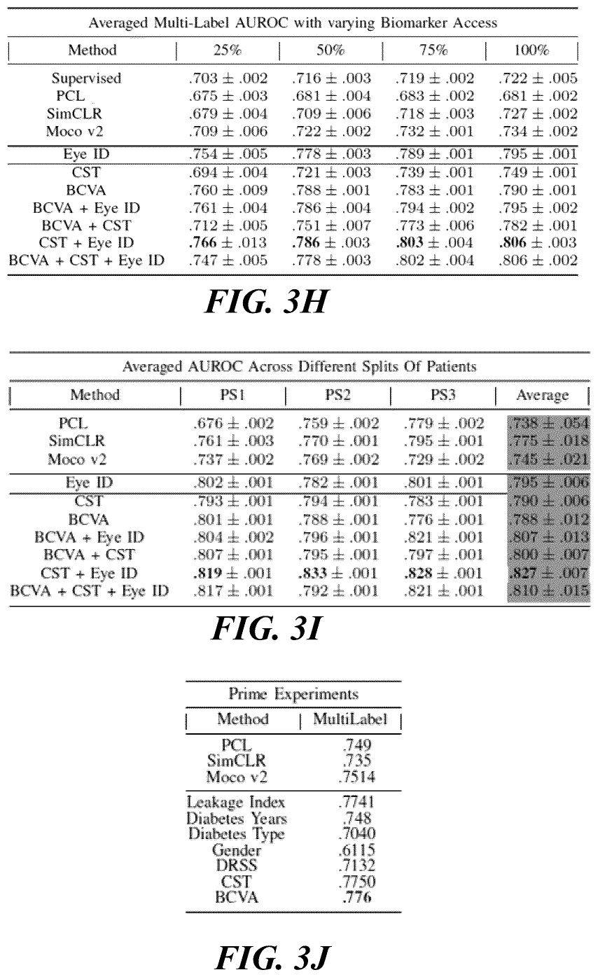

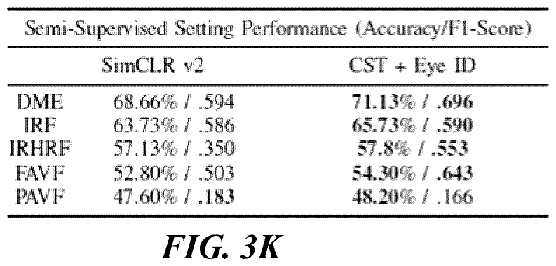

Method (e.g., 200a, 200b) then includes holding constant (212) the first weights of the AI model, expanding (214) the AI model with an additional portion (e.g., linear portion or a segmentation head), and adjusting (218) the second weights of the AI model via a contrastive loss function using the clinical labels in which the second dataset has a value of a presence of the medical condition in the meta data label. An example implementation is described in relation to FIGS. 3A.

The resulting classification engine 108 can then be used to generate an estimated/predicted a score 220 for a new data set 222 in a clinical application. In such embodiments, the classification engine 108 (shown as an example of a “Trained ML Model”) can additionally generate both an indication for a presence or non-presence of a disease or medical condition.

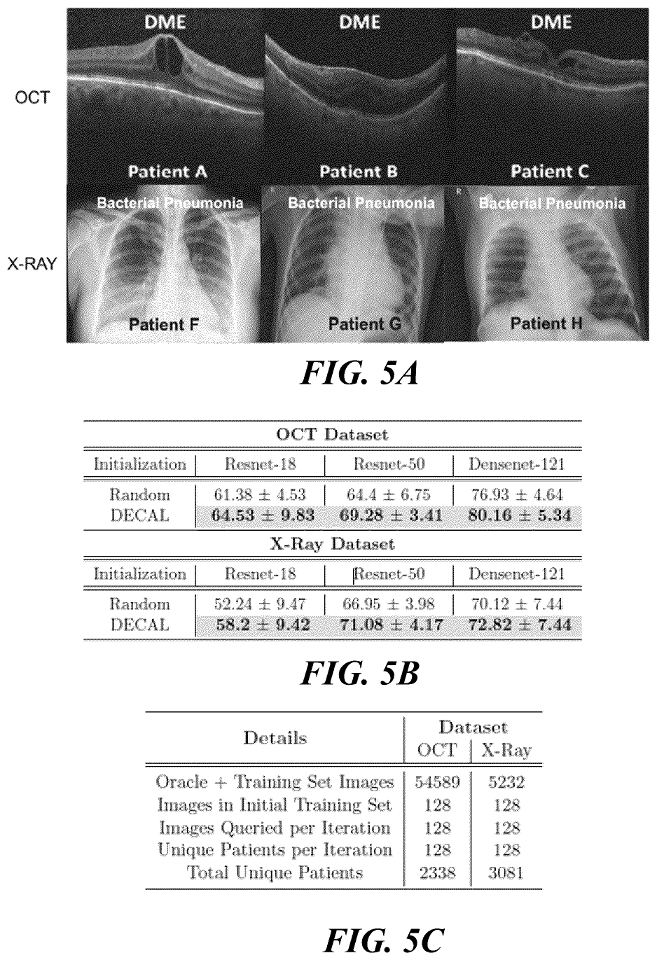

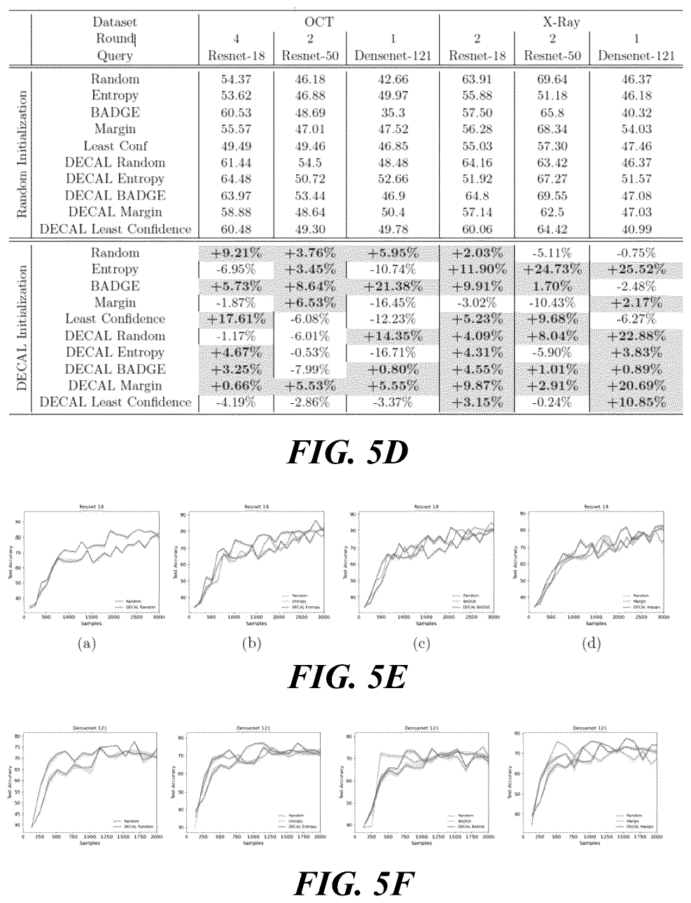

In FIG. 2B, Method 200b further, or alternatively includes computing (224) a distribution of a demographic identifier for a population set in the first training data set. Training 202 (shown as 202′) may be performed using the computed distribution by selecting the training data set according to the computed distribution. The demographic identifier can include age, gender, ethnicity, an indication that the patient is a smoker, or an indication that the patient regularly consumes alcohol. Demographic identifiers can include the presence of any disease or medical condition as would be included in a standardized electronic health record form. An example implementation is described in relation to FIGS. 5A-5F.

The classification engine 108, e.g., as described in relation to FIGS. 1. 2A, and 2B, as well as the trained ML model of the analysis module, can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, and depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Experimental #1—Asymmetric Multi-Modal Data Contrastive Learning Using Metadata in Clinical Data

A first study was conducted to develop the selection operation for a positive and negative data set in contrastive learning for medical images based on labels that can be extracted from clinical data. The selection operation can be applied to engineering images and various applications described herein. In the medical field, there exists a large pool of unlabeled images alongside a much smaller labeled subset. It is common that unlabeled images are only unlabeled with respect to certain specialized labels (e.g., biomarker labels). They often times have associated clinical data (e.g., metadata) that are generated as part of a standard visit to a medical practitioner. At least within the domain of ophthalmology, standard procedures of an eye exam may include collecting measured Best Central Visual Acuity (BCVA) and recording them in an eye exam chart when collecting images of the retina from Optical Coherence Tomography (OCT) scans.

Previous work in the medical field has shown these collected clinical values have correlations with structures that exist in OCT scans. The exemplary system and method can exploit these meta data relationships from clinical data for training data labeling, i.e., for biomarker classification. The exemplary system and method can employ the meta data in the clinical data as pseudo-labels for unlabeled data to choose positive and negative instances for training a backbone network with a supervised contrastive loss. The exemplary system and method can fine-tune a second network trained using the pseudo-labels for unlabeled data as biomarker labeled data in a second data set in a second modality, e.g., OCT scans. In the study, the exemplary system and method was observed to outperform standard supervised and state-of-the-art self-supervised methods by as much as 5% in terms of accuracy on individual biomarkers.

Methodology. FIG. 3A shows an exemplary method for supervised contrastive learning on a large, available first clinical data set that is then used to train a linear classifier on a second clinical data set. In FIG. 3A, within the large, available first clinical data, each individual image is associated with the clinical values (e.g., BCVA, CST) and eye identifiers that were taken during an original patient visit. The exemplary method employed in the first study used at least one of these clinical values to act as a label for each image in the dataset.

As shown in FIG. 3A, given an input batch of data xₖ, and clinical label yₖ, pairs (xₖ, yₖ)_{k=1, . . . , N}, the first study augmented the batch twice to get two copies of the original batch with 2N images and clinical labels. The augmentations were varied: randomly resized and cropped to a size of 224, randomly flipped horizontally, randomly adjusted for color jitter, and adjusted for data normalization. The process produced a larger set (xₗ, yₗ)_{l=1, . . . , 2N} that included two versions of each image that differ only due to the random nature of the applied augmentation without changing the general structure of the image. Thus, for every image xₖ and clinical label yₖ there exists two views of the image x₂ₖ and x₂ₖ₋₁ and two copies of the clinical labels that are equivalent to each other: y₂ₖ₋₁=y₂ₖ=yₖ.

As shown in FIG. 3A, supervised contrastive learning 302 was performed on the identified clinical label 304. The clinically labeled augmented batch was forward-propagated through an encoder network f(⋅) 306 that the example embodiment set to be the ResNet-18 architecture [60]. This resulted in a 512-dimensional vector rᵢ 308 that was sent through a projection network G(⋅) 310, which further compressed the representation to a 128-dimensional embedding vector zᵢ 312. G(⋅) 310 was chosen to be a multi-layer perceptron network with a single hidden layer as the projection network to reduce the dimensionality of the embedding before computing the loss and was discarded after training. A supervised contrastive loss was then performed on the output of the projection network 310 to train the encoder network 306.

FIG. 3A shows the supervised contrastive loss being used to train the encoder network with respect to a metadata label in the electronic medical record (e.g., BCVA label). To this end, embeddings with the same metadata label in the electronic medical record (e.g., BCVA label) were enforced to be projected closer to each other, while embeddings with differing metadata labels (e.g., BCVA labels) were projected away from each other.

The supervised contrastive loss function is provided by Equation 1.

$${L}_{{supcon}_{clinical}} = {\sum }_{i ∈ I} \frac{- 1}{\vert C ( i ) \vert} {\sum }_{c ∈ C ( i )} log \frac{exp ( {z}_{i} \cdot {z}_{c} / τ )}{{\sum }_{a ∈ A ( i )} exp ( {z}_{i} \cdot {z}_{a} / τ )} \qquad ( Eq . 1 )$$

In Equation 1, i is the index for the image of interest xᵢ. All positives c for image xᵢ were obtained from the set C(i), and all positive and negative instances a were obtained from the set A(i). Every element c of C(i) represented all other images in the batch with the same clinical label c as the image of interest xᵢ. zᵢ is the embedding for the image of interest; z_{c} represents the embedding for the clinical positives; zₐ represents the embeddings for all positive and negative instances in the set A(i). τ is a temperature scaling parameter that was set to 0.07 for all experiments. The loss function operated in the embedding space in which the goal was to maximize the cosine similarity between embedding zᵢ and its set of clinical positives z_{c}.

The loss function can enforce similarity between images with the same label and dissimilarity between images that have differing labels. Using the language of contrastive learning means that labels are used to identify the positive and negative pairs rather than augmentations. The loss is computed on each image xᵢ where i∈I=1, . . . , 2_{N} represents the index for each instance within the overall augmented batch. Each image xᵢ is passed through an encoder network f( ), producing a lower dimensional representation. The vector is further compressed through a projection head to produce the embedding vector zᵢ. Positive instances for image xᵢ come from the presence of a value for the meta data clinical label, and negative instances come from the non-presence of the meta data clinical label. The loss function operates in the embedding space where the goal is to maximize the cosine similarity between embedding zᵢ and its set of positives zₚ. The loss function defines images belonging to the same class through the use of clinical labels as a clinically aware supervised contrastive loss.

It was contemplated that set C(i) could represent any clinical label of interest. The first study used conventions to make the choice of clinical label in the loss transparent, e.g., a loss represented as L_{BCVA} indicated a supervised contrastive loss in which the label BCVA was utilized as the clinical label of interest. The first study determined that it was also possible to create an overall loss that is a linear combination of several losses on different clinical labels, e.g., Lₜₒₜₐₗ=L_{BCVA}+L_{CST} in which each clinical value, respectively, acted as a label for its respective loss.

After training the encoder with clinically supervised contrastive loss, the example embodiment moved to the second step 314 in FIG. 3A, in which the weights of the encoder 306 (shown as 306′) were frozen, and a linear layer 318 was appended to the output of the encoder 306′. The setup was trained on the OLIVES Biomarker dataset after biomarkers were chosen for the training. The linear layer was trained using cross-entropy loss to distinguish between the presence or absence of the biomarker of interest in the OCT scan. In this way, the exemplary method of the first study leveraged knowledge learned from training on clinical labels to improve performance on classifying biomarkers.

In the operation in part 2 of FIG. 3A, the biomarker of interest was chosen to be DME, and the input images to the network were labeled by the presence or absence of DME. The previously trained encoder with the supervised contrastive loss on the BCVA label from step 1 302 produced the representation for the input, and this representation is fine-tuned with the linear layer 318 to distinguish whether or not DME is present.

Interpretation. In [66], the authors present a theoretical framework for contrastive learning. Let X denote the set of all possible data points. In this framework, contrastive learning assumes access to similar data in the form of (x, x⁺) that comes from a distribution Dₛᵢₘ as well as k iid negative samples x₁⁻, x₂⁻, . . . xₖ⁻ from a distribution D_{neg}. The similarity is formalized through the introduction of a set of latent classes C and an associated probability distribution D_{c} over X for every class c∈C. D_{c}(x) quantifies how relevant x is to class c with a higher probability assigned to data points belonging to this class. Additionally, let ρ be defined as a distribution that describes how these classes naturally occur within the unlabeled data. From this, the positive and negative distribution are characterized as

$${D}_{s i m} = {𝔼}_{c \sim p} {D}_{c} ( x ) {D}_{c} ( {x}^{+} ) and {D}_{n e g} = {𝔼}_{c \sim p} {D}_{c} ( x ) {D}_{c} ( {x}^{-} )$$

where D_{neg} is from the marginal of Dₛᵢₘ.

The exemplary method differs from standard contrastive learning formulation due to a deeper look at the relationships between ρ, Dₛᵢₘ, and D_{neg}. In principal, during unsupervised training, there is no information that provides the true class distribution ρ of the dataset X. The goal of contrastive learning is to generate an effective Dₛᵢₘ and D_{neg} such that the model is guided towards learning ρ by identifying the distinguishing features between the two distributions.

Ideally, this guidance occurs through the set of positives belonging to the same class cp and all negatives belonging to any class cₙ≠cₚ as shown in the supervised framework [13]. Traditional approaches, such as [1A], [62], and [63], enforce positive pair similarity by augmenting a sample to define a positive pair that would clearly represent an instance belonging to the same class. However, these strategies do not define a process by which negative samples are guaranteed to belong to different classes. This problem is discussed in [63] where the authors decompose the contrastive loss Lᵤₙ as a function of an instance of a hypothesis class f∈F into Lᵤₙ(f)=(1−τ)L_{≠}(f)+(τ)L=(f). This states that the contrastive loss is the sum of the loss suffered when the negative and positive pair come from different classes (L_{≠}(f)) as well as the loss when they come from the same class (L₌(f)). In an ideal setting (L₌(f)) would approach 0, but this is impossible without direct access to the underlying class distribution ρ. However, it may be the case that there exists another modality of data during training that provides us with a distribution ρclin with the property that the KL(ρ_{clin}∥ρ)≤ϵ, where ϵ is sufficiently small. In this case, the Dₛᵢₘ and D_{neg} could be drawn from ρ_{clin} in the form:

$${D}_{s i m} = {𝔼}_{c \sim {p}_{clin}} {D}_{c} ( x ) {D}_{c} ( {x}^{+} ) and {D}_{n e g} = {𝔼}_{c \sim {p}_{clin}} {D}_{c} ( x ) {D}_{c} ( {x}^{-} ) .$$

If ρ_{clin} is a sufficiently good approximation for ρ, then there is a higher chance for the contrastive loss to choose positives and negatives from different class distributions and have an overall lower resultant loss.

In contrast, in the exemplary method, this related distribution that is in excess comes from the availability of clinical information within the unlabeled data and acts to form the ρ_{clin} that the method can use for choosing positives and negatives. This clinical data acts as a surrogate for the true distribution ρ that is based on the severity of disease within the dataset and thus has the theoretical properties discussed. There may exist many possible ρ_{clin}∈P_{clin}, where P_{clin} is the set of all possible clinical distributions. In the exemplary method, these clinical distributions can come from the clinical values of BCVA, CST, and Eye ID, which form the distributions ρ_{bcva}, ρ_{cst}, and ρ_{eyeid}.

Additionally, these distributions can be utilized in tandem with each other to create distributions of the form ρ_{bcva+cst}, ρ_{bcva+eye}, ρ_{cst+eye} and ρ_{bcva+cst+eye}.

Training. The first study took care to ensure that all aspects of the experiments remained the same, whether training was done via supervised or self-supervised contrastive learning on the encoder or cross-entropy training on the attached linear classifier. The encoder utilized was kept as a ResNet-18 architecture. The applied augmentations were random resize crop to a size of 224, random horizontal flips, random color jitter, and data normalization to the mean and standard deviation of the respective dataset. The batch size was set at 64. Training was performed for 25 epochs in every setting. A stochastic gradient descent optimizer was used with a learning rate of 1×10⁻³ and a momentum of 0.9.

Datasets. The Prime and TREX-DME studies provided a wealth of clinical information as part of their respective trials. In addition to the information provided by these studies, a trained grader in these studies performed interpretation on OCT scans for the presence of 20 different biomarkers including Intra-Retinal Hyper-Reflective Foci (IRHRF), Partially Attached Vitreous Face (PAVF), Fully Attached Vitreous Face (FAVF), Intra-Retinal Fluid (IRF), and Diffuse Retinal Thickening or Macular Edema (DRT/ME). The trained graders were blinded to clinical information whilst grading each of 49 horizontal SD-OCT B-scans of both the first and last study visit for each individual eye. The table of FIG. 3G shows clinical labels and biomarker labels available in the Prime and TREX-DME studies. In FIG. 3G, the ophthalmic labels were reproduced from [59], and the combination of the Prime and TREX-DME studies are referred to as the Olives Biomarker and Olives Clinial.

FIG. 3B shows images of biomarkers that were used in the first study. In FIG. 3B shows cross-sectional images of graded biomarkers, including Intra-Retinal Hyper-Reflective Foci (IRHRF), Intra-Retinal Fluid (IRF), Diabetic Macular Edema (DME), a Partially Attached Vitreous Face (PAVF), and a Fully Attached Vitreous Face (FAVF). In FIG. 3B, IRHRF indicated by the six white arrows are areas of hyperreflectivity in the intraretinal layers with or without shadowing of the more posterior retinal layers. IRF encompasses the cystic areas of hyporeflectivity. DME is the apparent swelling and elevation of the macula due to the presence of fluid. PAVF, indicated by an arrow, refers to the point of attachment. Additional descriptions of the biomarkers can be found in [2].

FIG. 3B additionally shows image 326 from the OLIVES Biomarker dataset labeled by the presence or absence of biomarkers (e.g., DME) and clinical metadata (e.g., Fluid IRF) that were fed into an encoder network trained by using BCVA values as the label as well as a SimCLR strategy. This produced an embedding for each image. These embeddings were visualized using t-SNE [61] with two components. It can be observed that from an encoder trained using BCVA labels with the supervised contrastive loss, the exemplary method can effectively achieve an embedding space that is separable with respect to biomarkers, while the standard contrastive learning method shows no separability for either of the biomarkers.

FIG. 3C and FIG. 3D show additional examples of OTC scans. Specifically, FIG. 3C shows Clinical and Biomarker labels associated with a single slice and a volume of OCT scans. FIG. 3D shows example images from OCT scans of pairs of images with the same BCVA value and different BCVA values across different patients. It can be observed that the same BCVA values corresponded with images that have more structural features in common.

In the first study, open adjudication was performed by an experienced retina specialist for difficult cases. The first study also introduced explicit biomarker labels to a subset of the data via a trained grader that performed interpretation on OCT scans for the presence of 20 different biomarkers. The trained grader was blinded to clinical information whilst grading each of the 49 horizontal SD-OCT B-scans of both the first and last study visit for each individual eye. Open adjudication was done with an experienced retina specialist for difficult cases. To this end, for each OCT scan labeled for biomarkers, there existed a one-hot vector indicating the presence or absence of 20 different biomarkers. The first study used the Intraretinal Hyperreflective Foci (IRHRF), Partially Attached Vitreous Face (PAVF), Fully Attached Vitreous Face (FAVF), Intraretinal Fluid (IRF), and Diffuse Retinal Thickening or Diabetic Macular Edema (DRT/ME) as the biomarkers.

When combining the datasets together, the first study focused on the clinical data that is commonly held by both datasets: BCVA, CST, and Eye ID. FIG. 3E shows the distributions within the OLIVES dataset for the clinical values of BCVA and CST (320, 322). FIG. 3E further shows a histogram 324 of eye/patient image distribution within the OLIVE data set. FIG. 3E shows the number of images with biomarker and clinical labels in the OLIVES dataset. In FIG. 3E, it can be observed that the distribution (320, 322) of BCVA and CST values across eyes and image quantities isn't noticeably biased towards any specific value. Rather, for each value, there is diversity in terms of the number of different eyes and number of images. In the case of biomarker information, there was no distribution problem because the identified images to be labeled were labeled in the same binary vector fashion for both datasets. FIG. 3G shows a summary of all of the data label availability with regard to clinical and biomarker information.

Together the Prime and TREX studies provided data from 96 unique eyes from 87 unique patients. The first study took 10 unique eyes from the Prime dataset and 10 unique eyes from the TREX dataset and used the data from these 20 eyes to create a test set. The remaining 76 eyes data was utilized for training in all experiments. To evaluate the model's performance in identifying each biomarker individually, a balanced test set for each biomarker was created by randomly sampling 500 images with the biomarker present and 500 images with the biomarker absent from the data associated with the test eyes.

Experiments and Metrics. During supervised contrastive training, a choice of a single clinical parameter or combination of parameters was chosen to act as labels. For example, in FIG. 3H, when the method is specified as BCVA, this indicated a supervised contrastive loss L_{BCVA}, where BCVA is utilized as the label of interest for the images in the dataset. Additionally, BCVA+CST refers to a linear combination of supervised contrastive losses that can be expressed as Lₜₒₜₐₗ=L_{BCVA}+LCST where each clinical value respectively acts as a label for its respective loss. A linear layer is then appended to this trained encoder and trained on the biomarker labels present in the OLIVES Biomarker dataset, consisting of approximately 7,500 images. This linear layer is trained on each biomarker individually, and accuracy, as well as F1-score in detecting the presence of each individual biomarker, is reported.

Performance is also evaluated in a multi-label classification setting where the goal is to correctly identify the presence or absence of all 5 biomarkers at the same time. While training in this multi-label setting, a binary cross-entropy loss across the multi-labeled vector was utilized. This is evaluated using the averaged area under the receiver operating curve (AUROC) over all 5 classes. This effectively works by computing an AUC for each biomarker and then averaging them. Additionally, we report the average precision and recall across all biomarkers.

Performance Results. The setup of the first study was compared against a fully supervised and fusion-supervised setting as well as state-of-the-art self-supervised contrastive learning frameworks. The fully supervised setting included standard cross-entropy training of the label of interest without any type of contrastive learning. Fusion supervised was the same as the fully supervised setting, except the clinical data for the associated image is appended to the last feature vector before the fully connected layer. The self-supervised frameworks were SimCLR [12], PCL [62], and Moco v2 [63].

Comparison with state of the art self-supervised. The first study evaluated the capability of the exemplary method to leverage a larger amount of clinical labels for performance improvements on the smaller biomarker subset in supervised contrastive training of the encoder network on the OLIVES Clinical dataset consisting of approximately 60,000 images. Table II shows that applying the exemplary method leads to improvements in the classification accuracy of each biomarker individually as well as an improved average AUROC score for detecting all 5 biomarkers concurrently when compared against the state-of-the-art self-supervised algorithms of interest.

The first study also observed visually in FIG. 3F how well training on a clinical label was performed in creating a separable embedding space for biomarkers. In FIG. 3F, a model was trained with a supervised contrastive loss on BCVA values using a test set labeled by the biomarkers DME and Intraretinal Fluid. The output embeddings for each image in the test set can be projected into a lower dimensional space with 1-D t-SNE [61]. For each class, the first study compared the mean and standard deviation on the generated t-SNE values. Using these parameters, the first study plotted in FIG. 3F a Gaussian curve for each class. It can be observed that the resulting representation can separate between present and absent forms of DME and Fluid IRF without having explicit training for these labels. The Gaussian distribution for each class appears clearly separated between the curve for the biomarker present and absent. This lends intuition that pre-training in the exemplary method with the clinical labels can effectively identify subsets of data that share features that should cluster together and thus act as a method to choose positives for a contrastive loss.

Performance of Self-Supervised Algorithms. Performance of the standard self-supervised methods appeared to be comparable to exemplary methods for IRF and DME but not for IRHRF, FAVF, and PAVF. It is contemplated that the exemplary method can identify positive instances that are correlated through having similar clinical metrics and, instead of over-reliance on augmentations from a single image, the exemplary method can find a more robust set of positive pairs that allows the model to more effectively identify fine-grained features of OCT scans.

Comparison with Supervised. A major challenge with detecting biomarkers is that they can be associated with small localized changes that exist within the overall OCT scan. IRHRF, FAVF, and PAVF are examples of biomarkers that fit this criteria. Biomarkers such as IRF and DME are more readily distinguishable as regions of high distortion caused by the presence of fluid within the retina slice. Because all biomarkers can potentially exist in the image at the same time, a model must be able to resolve small perturbations to distinguish biomarkers simultaneously. This can be especially difficult for traditional models that are likely to learn features of the easier-to-distinguish classes and be unable to identify the more difficult-to-find classes without sufficient training data [65]. To evaluate the impact of access to training data, the first study took the original training set of 7500 labeled biomarker scans and removed different-sized subsets. In FIG. 3H, each column represents the percentage amount of biomarker training data each method had access to. It can be observed that using the exemplary contrastive learning method can lead to improved performance, regardless of available training data.

It can also be observed that supervised methods that had access to the biomarker labels during the entirety of training were performing significantly worse as the training set was reduced. This may show the dependence that the methods have on a large enough training set because they are unable to leverage representations that may be learned from the large unlabeled pool of data. The self-supervised methods employed in the comparison were able to make use of the representations to perform better on the smaller amount of available training data but are still inferior to the exemplary method that integrates clinical labels into the contrastive learning process.

Performance with respect to individual clinical labels. Another aspect of the results in FIG. 3G is how well the used clinical labels correspond with the biomarker classification performance. In all cases, the results act as validation to the hypothesis that taking advantage of correlations that exist with certain clinical labels is beneficial for biomarker detection of individual OCT scans. However, from a medical perspective, certain outcomes would intuitively be more likely. For example, for IRF and DME, it makes sense that the best performance is associated with using CST values because CST tends to increase or decrease depending on the severity of IRF and DME. Further analysis showed that combinations of clinical labels with a linear combination of losses, such as BCVA+Eye ID and BCVA+CST. lead to performance improvements in FAVF, IRHRF, and Multilabel classification. A reason for this is that each clinical value can be thought of as being associated with its own distribution of images. By having a linear combination of losses on two clinical values, we are effectively choosing positive instances from closely related but slightly varying distributions.

FIG. 3E shows the distributions. It can be observed that because BCVA and CST have different ranges of potential values, as observed along the x-axis of this figure, this means that for any individual values there is a different number of associated eyes and images. Effectively, this means that there is varying diversity with respect to any individual label. This may allow the model to better learn features relating to contrasts that each respective distribution is able to observe. This improves robustness in identifying the more difficult biomarkers, which may be the reason for overall improvement in the multi-label classification task.

Prime Clinical Experiments. FIG. 3J shows performance data based on averaged AUCROC after training the encoder using data from the Prime dataset. The first study trained the encoder using just the 29000 images in the Prime dataset, which has a wider variety of clinical information that the study can potentially use as a label. In addition to BCVA, CST, and Eye ID that are available across both the Prime and TREX datasets, there are clinical parameters that exist for the Prime dataset specifically, such as the type of diabetes of the patient, the diabetic retinopathy severity score (DRSS), and various demographic information.

In FIG. 3J, it can be observed that CST and BCVA generally outperformed all other methods, but there are certain modalities that perform better than the self-supervised baselines. FIG. 3J also shows that the exemplary method can perform satisfactorily even with the constraints imposed by using the Prime dataset alone.

Semi-Supervised Experiments. The first study also compared the exemplary method within a state-of-the-art semi-supervised framework (see FIG. 3K). In this experiment, the first study followed the setting of [44]. To do this, the first study took an encoder pre-trained with a contrastive learning strategy and fine-tuned it with a linear layer with only 25% of available biomarker data for each biomarker in the study. The model then becomes the teacher model that the study used to train a corresponding student model. The student model has access to both the 25% subset the teacher was trained on as well as the remaining biomarker data that the study designated to be the unlabeled subset. The teacher was used to provide logit outputs that are then used as part of a distillation loss discussed in [44] to train the student model. In this way, the study modeled the semi-supervision setting by making use of a small amount of labeled data for the teacher model and then both labeled and unlabeled data for the student. The study compared the performance of this setup on a pretraining with respect to SimCLR and our combined clinical contrastive strategy that makes use of both the CST and Eye ID label distributions. The study observed in FIG. 3K that the exemplary method consistently outperformed the model that used SimCLR pre-training.

### Discussion

Biomarkers refer to “any substance, structure, or process that can be measured in the body or its products and influence or predict the incidence of outcome or disease [1].” In order to detect and treat disease, the evaluation of biomarkers is a necessary step in any clinical practice [2]. However, the interpretation of biomarkers from imaging data is a time-consuming and expensive process. In a clinical setting, the interpretation demands of experts have grown disproportionately relative to available staff. A study from 2015 [3] showed that radiologists are tasked with interpreting 16.1 images per minute, which has contributed to fatigue, burnout, and an increased error-rate. Given the importance of biomarkers and their difficulty in acquisition, it is natural to invest in the development of machine learning algorithms to automate the detection of key biomarkers directly from their associated imaging modality. Accomplishing this goal would assist clinical practitioners in making better treatment decisions with the goal of arriving at more favorable outcomes for their patients. In order to bring this technology to fruition, acquiring access to large quantities of labeled examples is a necessary step to train any conventional deep learning architecture [4]. Obtaining such a dataset is a major bottleneck because labels for medical data are expensive and time-consuming to curate due to the aforementioned difficulties with interpretation.

Even though biomarker labels are hard to obtain, there are other types of measurements that are taken as part of standard visits to the clinical practitioner that are typically easier to obtain in large quantities. They are termed clinical labels. The present disclosure utilizes the correlations present in the larger corpus of clinically labeled data, e.g., to improve biomarker detection performance for indicators of the disease Diabetic Retinopathy (DR) within the setting of Optical Coherence Tomography (OCT) scans.

Biomarkers such as Intraretinal Fluid (IRF), Diabetic Macular Edema (DME), and Intra-Retinal Hyper-Reflective Foci (IRHRF) in FIG. 3B are direct indicators of (DR). Other biomarkers, such as Partially and Fully Attached Vitreous Face (PAVF and FAVF) are not direct indicators of disease, but detecting them can potentially be used to identify certain complications [5]. These specific biomarkers are visible on OCT scans, as shown in FIG. 3B. Even though acquiring access to these biomarkers is difficult, there are oftentimes associated clinical labels taken during routine care with an ophthalmologist. Identifying information such as the patient's identity, type of diabetes, and amount of time with diabetes are all easily obtained as part of a reporting process [6]. Best Corrected Visual Acuity (BCVA) and Central Subfield Thickness (CST) are both collected as part of standard clinical exam procedures. BCVA is measured from eye exam charts, and CST values can be processed directly from values obtained from an OCT machine. Visually, it can be observed from FIG. 3 that OCT scans with the same BCVA values exhibit more common structural characteristics than scans with different BCVA values. Studies such as [7]-[10] confirm that these measured clinical labels can act as indicators of structural changes that manifest themselves in OCT scans, as well as the severity of DR associated with the patient. These works indicate that the clinical labels that are collected in abundance during standard clinical practice exhibit non-trivial relationships with key biomarkers of DR.

The example embodiments described herein make use of these correlations that exist in clinical data in order to improve biomarker detection performance. In particular, the example embodiment addresses this detection by using a contrastive learning approach [11] that incorporates clinical labels into the deep learning framework. Contrastive learning is a methodology that functions by creating a representation space by minimizing the distance between positive pairs and maximizing the distance between negative pairs of images. Traditional contrastive learning approaches, such as [12], generate positive pairs from augmentations of a single image and treat all other images in the batch as the negative pairs.

However, from a medical imaging point of view, arbitrary augmentations, like in [12], have the potential to occlude the small localized regions where biomarkers may be present. The authors in [13] choose positive pairs within the same class label and negative pairs are from all other classes. However, in our setting, the supervised biomarker-labeled data is insufficient to perform supervised contrastive learning due to the relatively scarce amount of available data. Hence, state of the art contrastive learning techniques that perform well on natural image datasets may not be applicable to medical data as will be illustrated in this study.

Embodiments of the present disclosure include supervised contrastive learning by utilizing clinical labels to discriminate between positive and negative pairs of images. This allows the model to learn a representation space that can effectively separate embeddings of OCT scans into semantically interpretable groups by enforcing images with similar BCVA values, CST values, or images from the same eye to be close to each other in the representation space. These representations will then be utilized to train a linear classifier utilizing a much smaller subset of biomarker labels. As a result, the model will be able to utilize the larger pool of clinical labels in order to better learn how to classify specific biomarkers. The first study showed (i) that utilizing clinical labels associated with OCT scans to train an effective supervised contrastive learning framework and (ii) that the exemplary method can outperform traditional approaches that use direct supervision on biomarker labels as well as state-of-the-art self-supervised strategies. The first study provided a comprehensive study on clinical label usage and their effects on biomarker identification.

Contrastive learning refers to a family of self-supervised algorithms that leverages differences and similarities between data points in order to extract useful representations for downstream tasks. The basic premise is to train a model to produce a lower dimensional space where similar pairs of images (positives) project much closer to each other than dissimilar pairs of images (negatives).

Contrastive learning approaches such as [1A], [2A], and [3A] can generate positive pairs of images through various types of data augmentations such as random cropping, multi-cropping, and different types of blurs and color jitters. A classifier can then be trained on top of these learned representations while requiring fewer labels for satisfactory performance. Recent work has explored the idea of using medically consistent meta-data as a means of finding positive pairs of images alongside augmentations for a contrastive loss function. [4A] showed that using images from the same medical pathology as well as augmentations for positive image pairs could improve representations beyond standard self-supervision. [5A] demonstrated utilizing contrastive learning with a transformer can learn embeddings for electronic health records that can correlate with various disease concepts. [6A] investigated choosing positive pairs from images that exist from the same patient, clinical study, and laterality. These works demonstrate the potential of utilizing clinical data within a contrastive learning framework. However, these methods were tried on limited clinical data settings, such as choosing images from the same patient or position relative to other tissues. In contrast, embodiments of the present disclosure can explicitly use measured clinical labels as its own label for training a model. By doing this, embodiments of the exemplary method can provide a comprehensive assessment of what kinds of clinical data can possibly be used as a means of choosing positive instances.

OCT Datasets. Previous OCT datasets for machine learning have labels for specific segmentation and classification tasks regarding various retinal biomarkers and conditions. [50] contains OCT scans for classes of OCT disease states: Healthy, Drusen, DME, and choroidal neovascularization (CNV). [51] and [52] introduced OCT datasets for the segmentation of regions with age-related macular degeneration (AMD). [53] created a dataset for the segmentation of regions with DME. In all cases, these datasets do not come with associated comprehensive clinical information nor a wide range of biomarkers to be detected.

The exemplary method and system can build on these clinical studies to add explicit biomarker information to a subset of this data. In this way, the exemplary method and system can curate a novel dataset that allows experimentation of OCT data from the perspective of both clinical and biomarker labels.

Incorporating Clinical Data with Multi-Modal Learning. A survey of radiologists [14] showed that access to clinical labels had an impact on the quality of interpretation of images. However, standard deep learning architectures only utilize visual scans without contextualization of other clinical labels. This has motivated research into different ways of incorporating clinical labels into the deep learning framework. One approach that has gained traction is to treat clinical labels as its own feature vector and then fuse this with the features learned from a CNN on associated image data. [15] showed how combining image features from a centigram with clinical records such as PH-value, HPV signal strength, and HPV status could be utilized to train a network to diagnose cervical dysplasia. [16] incorporated data from a neurophysical diagnosis with features from MRI and PET scans for Alzheimer's detection. [17] combined image information along with skin lesion data such as lesion location, lesion size, and elevation for the task of basal cell carcinoma detection. Similarly, [18] utilized macroscopic and dermatoscopic data along with patient meta data for improved skin lesion classification. [19] performed a fusion of EMR datasets with various information such as diagnosis, prescriptions, and medical notes for the task of dementia detection. Other works have performed multi-modal fusion between different types of imaging domains. [20] fused images from CT, MRI, and PET to show how each can provide different types of information for clinical treatment. [21] fused data from PET and MRI scans for the diagnosis of Alzheimer's disease. [22] incorporated imaging data along with genomics data for lung cancer recurrence prediction. Each of these works are similar to ours in the sense that they each are trying to make use of available clinical data. While these methods have shown improved performance in certain applications, they have disadvantages that stem from their method of using clinical labels. By using only an additional clinical feature vector associated with already labeled data, these frameworks do not provide a means to incorporate the large pool of unlabeled data into the training process.

In contrast, the exemplary system and method of the first study used clinical data within a contrastive learning operation to incorporate unlabeled data into the training process while leveraging the clinical intuition provided by the available meta data information.

Deep Learning and OCT. A desire to reduce diagnosis time and improve timely accurate diagnosis has led to applying deep learning ideas to detecting pathologies and biomarkers directly from OCT slices of the retina. Early work involved a binary classification task between healthy retina scans and scans containing age-related macular degeneration [23]. [24] introduced a technology to do relative afferent pupillary defect screening through a transfer learning methodology. [25] showed that transfer learning methods could be utilized to classify OCT scans based on the presence of key biomarkers. [26] showed how a dual-autoencoder framework with physician attributes could improve classification performance for OCT biomarkers. [27] analyzed COVID-19 classification in neural networks to analyze and explain deep learning performance. Subsequent work from [28] showed that semantic segmentation techniques could identify regions of fluid that are oftentimes indicators of different diseases. [29] expanded previous work towards the segmentation of a multitude of different biomarkers and connected this with referral for different treatment decisions. [30] showed that segmentation could be done in a fine-grained process by separating individual layers of the retina. Other work has demonstrated the ability to detect clinical information from OCT scans, which is significant for suggesting correlations between different domains. [31] showed that a model trained entirely on OCT scans could learn to predict the associated BCVA value. Similarly, [32] showed that values such as retinal thickness could be learned from retinal fundus photos. All these methods demonstrate the potential for deep learning within the medical imaging domain in the presence of a large corpus of labeled data. On OCT scans, where this assumption cannot always be made, contrastive learning methods have grown in popularity. None of the references address the issue noted herein nor provide the disclosed exemplary method and system.

Other Contrastive Learning Approaches. Contrastive learning [11] refers to a family of self-supervised methods that make use of pre-text tasks or embedding enforcement losses with the goal of training a model to learn a rich representation space without the need for labels. The general premise is that the model is taught an embedding space where similar pairs of images project closer together, and dissimilar pairs of images are projected apart. Approaches such as [12]. [33]-[35] all generate similar pairs of images through various types of data augmentations such as random cropping, multi-cropping, and different types of blurs and color jitters. A classifier can then be trained on top of these learned representations while requiring fewer labels for satisfactory performance. The authors in [65] augment contrastive class-based gradients and then train a classifier on top of the existing network. The authors in [27] augment contrastive class-based gradients and then train a classifier on top of the existing network. Other work [36], [37] used a contrastive learning setup with a similarity retrieval metric for weak segmentation of seismic structures. [38] used volumetric positions as pseudo-labels for a supervised contrastive loss. Hence, contrastive learning presents a way to utilize a large amount of unlabeled data for performance improvements on a small amount of labeled data.

Although the aforementioned works have been effective in natural images and other applications, natural image-based augmentations and pretext tasks are insufficient for OCT scans. [39] introduced a pretext task that involved predicting the time interval between OCT scans taken by the same patient. [40] showed how a combination of different pretext tasks, such as rotation prediction and jigsaw re-ordering can improve performance on an OCT anomaly detection task. [41] showed how assigning pseudo-labels from the output of a classifier can be used to effectively identify labels that might be erroneous. These works all identify ways to use variants of deep learning to detect important biomarkers in OCT scans. However, they differ fundamentally from the exemplary system and method of the first study in that they don't utilize the abundance of clinical data to aid in the training of a model.

The literature on self-supervised learning has shown that while it is possible to leverage data augmentations as a means to create positive pairs for a contrastive loss, this is often not so simple within the medical domain due to issues with the diversity of data and small regions corresponding to important biomarkers. Previous work has shown that it is possible to use contrastive learning with augmentations on top of an Imagenet [42] pretrained model to improve classification performance for x-ray biomarkers [43]. However, this is sub-optimal in the sense that the model required supervision from a dataset with millions of labeled examples. As a result, recent work has explored the idea of using medically consistent meta-data as a means of finding positive pairs of images alongside augmentations for a contrastive loss function. [44] showed that using images from the same medical pathology as well as augmentations for positive image pairs could improve representations beyond standard self-supervision. [45] demonstrated utilizing contrastive learning with a transformer can learn embeddings for electronic health records that can correlate with various disease concepts. Similarly, [46] utilized pairings of images from X-rays with their textual reports as a means of learning an embedding for the classification of various chest X-ray biomarkers. [47] investigated choosing positive pairs from mages that exist from the same patient, clinical study, and laterality. [48] used a contrastive loss to align textual and image embeddings within a chest X-ray setting. [49] incorporated a contrastive loss to align embeddings from different distributions of CT scans. These works demonstrated the potential of utilizing clinical data within a contrastive learning framework. However, these methods were performed on limited clinical data settings, such as choosing images from the same patient or position relative to other tissues.

In contrast, the exemplary system and method improve on these systems by explicitly using measured clinical labels (e.g., from an eye-disease setting) as its own label for training a model. In doing this, the exemplary system and method can provide a comprehensive assessment and usage of metadata clinical data in electronic medical records as a means of choosing positive instances from the perspective of medical image scans (e.g., OCT scans) using the application of the supervised contrastive loss function.

### Experiment #2—Asymmetric Multi-Modal Data Contrastive Learning Using Metadata in Engineering Data

In seismic interpretation, pixel-level labels of various rock structures can be time-consuming and expensive to obtain due to a reliance on an expert interpreter. As a result, there oftentimes exists a non-trivial quantity of unlabeled data that is left unused simply because traditional deep learning methods rely on access to fully labeled volumes.

An exemplary method and system are disclosed that employ contrastive learning for semantic segmentation of rock volumes using unlabeled data. The contrastive learning defines positive and negative pairs of images to utilize within a contrastive loss.

In this study, the exemplary method and system choose positives to assign as positional labels to cross-lines that are adjacent to each other within a seismic volume. From these assigned labels, a supervised contrastive loss is used to train an encoder network to learn the distinguishing characteristics of seismic data from a contrastive loss. Training in this manner led to a representation space more consistent with the seismic setting and was shown to outperform a state-of-the-art self-supervised methodology in a semantic segmentation task.

Contrastive learning approaches have been proposed that use a self-supervised methodology in order to learn useful representations from unlabeled data. However, traditional contrastive learning approaches are based on assumptions from the domain of natural images that do not make use of seismic context. The exemplary method and system employ a positive pair selection strategy based on the position of slices within a seismic volume.

Dataset. For all of our experiments, the second study utilized a publicly available F3 block located in the Netherlands (Alaudah et al., 2019a). The dataset contains full semantic segmentation annotations of the rock structures present. The second study utilized the training and test sets introduced by the original author. The training volume included 400 in-lines and 700 cross-lines. The 700 cross-lines were used for training. The test set included data from two neighboring volumes. This first volume included 600 labeled in-lines and 200 labeled cross-lines. The second volume included 200 in-lines and 700 cross-lines. For testing, the second study combined the cross-lines from each volume to form a larger 900 crossline test set. These 900 images were divided into three test splits consisting of 300 images each. The results show the average mean intersection over union across each of these test splits.

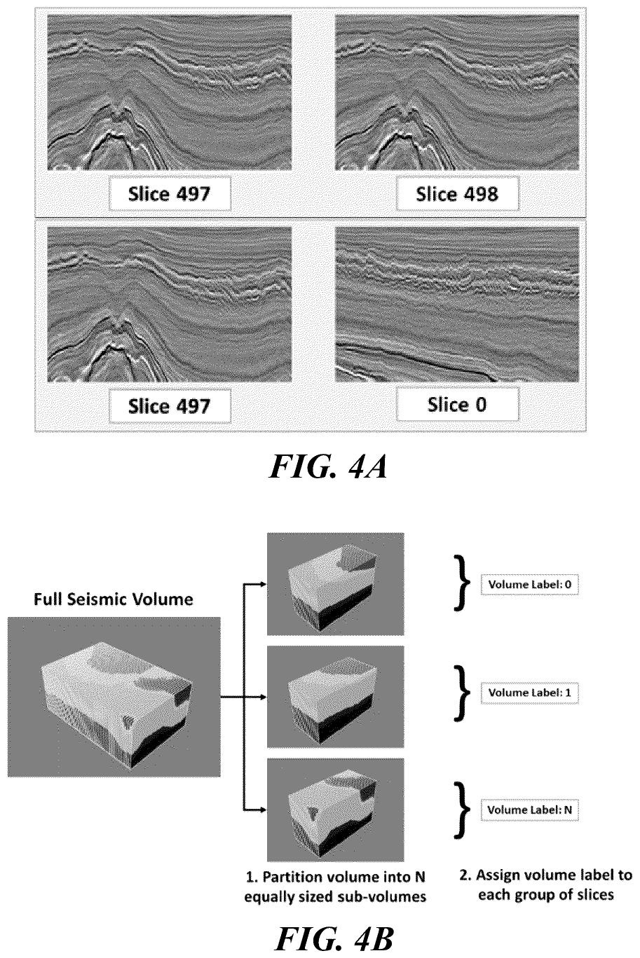

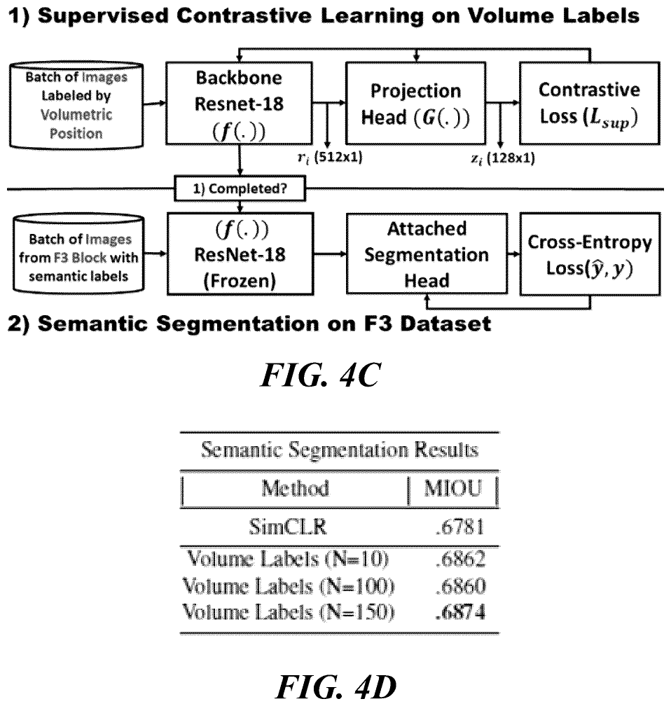

Volume-Based Labels. To select better positive pairs for a contrastive loss, the second study assigned pseudo-labels to cross-lines based on their position within the volume. FIG. 4B shows the process. From the starting set of 700 cross-lines, the second study defined a hyperparameter N that dictates the number of equally sized partitions that will divide the volume. If N=100, then the volume will be divided into 100 equally sized partitions consisting of 7 cross-lines each. After dividing the volume in this manner, cross-lines belonging to the same sub-volume are assigned the same volume position label V_{L}. The volume label acts to identify cross-lines that are more likely to share structural features in common due to being next to each other within the F3 block.

Supervised Contrastive Learning Framework. Once the volume labels (V_{L}) are assigned, the second study utilized the supervised contrastive loss to bring embeddings of images with the same volume label together and push apart embeddings of images with differing volume labels. FIG. 4C shows a flowchart of the overall setup.

In FIG. 4C, the system first pre-train a backbone ResNet-18 model (He et al., 2016) using the volume labels to identify positive and negative pairs of images for the supervised contrastive loss. Each cross-line image xᵢ is passed through the ResNet-18 encoder network f( ) 402, producing a 512×1 dimensional vector rᵢ. The vector is further compressed through a projection head G(:), which is set to be a multi-layer perceptron with a single hidden layer. The projection head is used to reduce the dimensionality of the representation and is discarded after training. The output of G(:) is a 128×1 dimensional embedding zᵢ. In this embedding space, the dot product of images with the same volume label (the positive samples) are maximized, and those with different volume labels (the negative samples) are minimized. This takes the form of equation 1 where positive instances for image xi come from the set P(i), and positive and negative instances come from the set A(i). zᵢ and zₚ are embeddings that originated from each of these sets, respectively. τ is a temperature scaling parameter set to 0.07 for all experiments.

$${L}_{sup} = {\sum }_{i ∈ I} \frac{- 1}{\vert P ( i ) \vert} {\sum }_{p ∈ P ( i )} log \frac{exp ( {z}_{i} \cdot {z}_{p} / τ )}{{\sum }_{a ∈ A ( i )} exp ( {z}_{i} \cdot {z}_{a} / τ )} \qquad ( Eq . 2 )$$

After pre-training the network via the supervised contrastive loss on volume position labels, the system moves to step two in the methodology. In this step, the weights of the previously trained encoder are frozen and a semantic segmentation head from the Deep Lab v3 architecture (Chen et al., 2018) is appended to the output of the encoder.

The second study passed batches of images from the same 700 cross-lines that were used in the previous step but now re-introduce the associated semantic segmentation labels for each cross-line. The output of the head is a pixel-level probability vector map ŷ that is used as input to a cross-entropy loss with the ground truth segmentation labels y. The loss function is used to train the segmentation head to segment the volume into relevant rock structure regions. The exemplary method can fine-tune the semantic segmentation head using the representations learned from the contrastive loss.

Results. The study compared the representations learned from the exemplary contrastive learning strategy perform relative to representations learnt from other methods, e.g., Sim-CLR (Chen et al., 2020). The architectures were kept constant as ResNet-18 for both experiments. Augmentations for both methods during the contrastive training step involved random resize crops to a size of 224, horizontal flips, color jittering, and normalization to the mean and standard deviation of the seismic dataset. During the training of the segmentation head, augmentations were limited to just the normalization of the data.

The batch size was set to 64. Training was performed for 50 epochs for both the contrastive pre-training as well as the segmentation head fine-tuning. A stochastive gradient descent optimizer was utilized with a learning rate of 0.001 and a momentum of 0.9. The second study assessed the quality of our method through the average mean intersection over the union metric of the three test splits we introduced.

FIG. 4D shows a summary of the comparative results. In FIG. 4D, it can be observed that regardless of the number of partitions in which the volume is divided, the exemplary method appears to outperform the state-of-the-art SimCLR framework.

By varying the partition hyper-parameter N for a lower value of N that provides a higher number of partitions, the contrastive loss exhibited stronger correlations with each other.

### Discussion

During exploration for oil and gas, seismic acquisition technology outputs a large amount of data in order to obtain 2D and 3D images of the surrounding subsurface layers. Despite the potential advantages that come with access to this huge quantity of data, processing and subsequent interpretation remain a major challenge for these companies. Interpretation of seismic volumes is done in order for geophysicists to identify relevant rock structures in regions of interest. Conventionally, these structures are identified and labeled by trained interpreters, but this process can be expensive and labor-intensive. This results in the existence of a large amount of unlabeled data alongside a smaller number that has been fully interpreted. To overcome these issues, work has gone into using deep learning to automate the interpretation process.

However, a major problem with any conventional deep learning setup is the dependence on having access to a large pool of training data. This dependency is not reliable within the context of seismic. To overcome this reliance on labeled data as well as leverage the potentially larger amount of unlabeled data, contrastive learning has emerged as a promising research direction. The goal of contrastive learning approaches is to learn distinguishing features of data without needing access to labels. This is done through algorithms that learn to associate images with similar features (positives) together and disassociate images with differing features (negatives). Traditional approaches can do this by taking augmentations from a single image and treating these augmentations as the positives, while all other images in the batch are treated as the negative pairs. These identified positive and negative pairs are inputted into a contrastive loss that minimizes the distance between positive pairs of images and maximizes the distance between negative pairs in a lower dimensional space. These approaches work well within the natural image domain but can exhibit certain flaws within the context of seismic imaging.

Naive augmentations, for example, could potentially distort the textural elements that constitute different classes of rock structures. A better approach for identifying positive pairs of images would be by considering the position of instances within the volume. FIG. 4A shows that seismic images that exist closer to each other in a volume can exhibit more structural components in common than those that are further apart. Therefore, these images that are closer to each other within a volume have similar features that a contrastive loss would be able to distinguish from features of other classes of rocks.

The second study took advantage of the correlations between images close to each other in a volume through a contrastive learning methodology. Specifically, the second study partitions a seismic volume during training into smaller subsets and assigns the slices of each subset the same volume-based label. The second study utilized volume-based labels to train an encoder network with a supervised contrastive loss (Khosla et al., 2020). Effectively, this means that the model is trained to learn to associate images close in the volume together and disassociate images that are further apart. From the representation space learned by training in this manner, we fine-tune an attached semantic segmentation head using the available ground truth labels.

The original usage of deep learning for seismic interpretation tasks was within the context of supervised tasks (Di et al., 2018), where the authors performed salt-body delineation. Further work into supervised tasks included semantic segmentation using deconvolution networks (Alaudah et al., 2019a). Deep learning was also utilized for the task of acoustic impedance estimation (Mustafa et al., 2020; Mustafa and AlRegib, 2020). However, it was quickly recognized that labeled data is expensive, and training on small datasets leads to poor generalization of seismic models. For this reason, the research focus switched to methods without as high of dependence on access to a large quantity of labeled data. This includes (Alaudah and AlRegib, 2017; Alaudah et al., 2019b, 2017), where the authors introduced various methods based on weak supervision of structures within seismic images. Other work introduced semi-supervised methodologies, such as (Alfarraj and AlRegib, 2019), for the task of elastic impedance inversion. (Lee et al., 2018) introduced a labeling strategy that made use of well logs alongside seismic data. (Shafiq et al., 2018a) and (Shafiq et al., 2018c) introduced the idea of leveraging learned features from the natural image domain. Related work (Shafiq et al., 2022) and (Shafiq et al., 2018b) showed how saliency could be utilized within seismic interpretation. More recent work involves using strategies such as explainability (Prabhushankar et al., 2020) and learning dynamics analysis (Benkert et al., 2021).

Despite the potential of pure self-supervised approaches, there isn't a significant body of work within the domain of seismic. Work such as (Aribido et al., 2020) and (Aribido et al., 2021) showed how structures can be learned in a self-supervised manner through manipulation of the latent space. (Soliman et al., 2020) created a self and semi-supervised methodology for seismic semantic segmentation. More recent work (Huang et al., 2022) introduced a strategy to reconstruct missing data traces. The most similar work to ours occurs within the medical field where (Zeng et al., 2021) uses a contrastive learning strategy based on slice positions within an MRI and CT setting. The exemplary method of the second study differs from previous works in using contrastive learning strategy based on volume positions within a seismic setting.

### Experiment #3

Conventional machine learning systems that operate on natural images assume the presence of attributes within the images that lead to some decisions. However, decisions in the medical domain are a result of attributes within medical diagnostic scans and electronic medical records (EMR). Hence, active learning techniques that are developed for natural images are insufficient for handling medical data. By reducing this insufficiency by designing a deployable clinical active learning (DECAL) framework within a bi-modal interface so as to add practicality to the paradigm, the exemplary system and method can be implemented as a plug-in method that makes natural image-based active learning algorithms generalize better and faster.

It was observed that on two medical datasets on three architectures and five learning strategies, DECAL can increase generalization across 20 rounds by approximately 4.81%. DECAL leads to a 5.59% and 7.02% increase in average accuracy as an initialization strategy for optical coherence tomography (OCT) and X-Ray, respectively. The active learning results could achieve using 3000 (5%) and 2000 (38%) sampl7es of OCT and X-Ray data respectively.

Experiment. The third study conducted a set of controlled experiments to evaluate the effectiveness of a DECAL framework relative to conventional frameworks. The study used images and EMR data from the OCT dataset by Kermany et al. (2018). The dataset included grayscale, cross-sectional, and foveal scans having varying sizes. The third study used images from 3 retinal diseases: 10488 choroidal neovascularization (CNV), 36345 diabetic macular edema (DME) and 7756 Drusen annotated at the image level. Samples in training and oracle sets were from 1852 unique patients. The test set included 250 images from each diseased class from 486 unique patients.

The study also used images and EMR data from the X-Ray dataset also by Kermany et al. (2018). The X-rays were grayscale, cross-sectional chest scans from children belonging to a healthy class and 2 types of pneumonia: viral and bacterial annotated at the image level.

The study used 1349 healthy, 1345 viral, and 2538 bacterial samples in the combined training and oracle sets from 2650 unique patients. The test set included 234 healthy, 148 viral, and 242 bacterial images from 431 unique patients. There was also no overlap in patients or imagery in train or test sets for both datasets. This meant the imagery in the train and test sets came from different patient cohorts. EMR data used for our analysis was patient identity from both datasets.

Active Learning with EMR Data. FIG. 5A shows sample scans from each dataset with patients having the same disease. In FIG. 5A, the visual characteristics across patients are noticeably different. Intra-class diversity is a typical occurrence in medical datasets. Existing active learning paradigms often fail to properly account for disease manifestations when there is less data, which can be dangerous in critical domains like medicine.

The third study posited that EMR data, in the form of patient identity, can be leveraged to account for the intra-class diversity present in medical datasets. The third study used patient identity as a plug-in constraint that can be applied prior to sample selection with any query acquisition function. The next batch of informative samples will have a unique patient identity from the unlabeled pool and be appended to the training set. This process is repeated to determine the minimum number of labeled samples needed to maximize model performance.

The third study assessed the active learning framework on Resnet-18, Resnet-50, and Densenet-121 (He et al. (2016); Huang et al. (2017)). The third study did not use pre-trained models in any of the analyses. The third study used the Adam optimizer with a learning rate of 1.5e-4. Hyper-parameters were tuned based on the OCT dataset, and then the same parameters were used for the X-Ray dataset. For each round, the Resnet and Densenet models were trained until 98% and 94% accuracy were achieved on the training set, respectively. Following each round, the model's weights were reset and randomly initialized. This was repeated with three different random seeds. The study aggregated and reported average accuracy and standard deviation. All images are resized to 128×128, and OCT scans were normalized with μ=0.1987 and σ=0.0786 while X-Rays with μ=0.4823 and σ=0.0379.

FIG. 5C shows implementation details for each dataset. FIG. 5C also shows the number of samples added after each training round.

Initializing Active Learning with EMR Data. Existing frameworks typically start active learning by randomly selecting a small number of samples to train the initial model. Subsequently, they apply methods of ranking sample informativeness. By doing this, they naively assume that the data distribution is even, which may not be the case in medical datasets, as shown in FIG. 5A. Randomly selecting from an unbalanced distribution is not guaranteed to gather a representative sample of the classes present (Zhu et al. (2008)).

The third study employed the integration of EMR data from the outset to circumvent this issue by first computing the distribution of patients throughout the unlabeled pool. Then, the third study selected a fixed number of images from unique patient identifiers and paired them with their annotation for the initial training set. The intuition behind this strategy is for the first training samples to have maximally dissimilar images. The samples can then be used to start the DECAL operation or analysis.

The third study evaluated two experimental modalities in the initialization phase depending on the availability of data: a large training data set and a small training data set.

Large Initial Training Set. The third study selected 1000 samples at random from the unlabeled pool and trained a model for each architecture and dataset for the first round only as our baseline. Then, the study performed DECAL initialization by selecting one image from 1000 unique patients in the unlabeled pool. The study then trained a model for each architecture and dataset for the first round only and compared it to the baseline by reporting the average accuracy and standard deviation on the test set. FIG. 5B shows the results.

Small Initial Training Set. The study selected 128 samples with DECAL initialization then started both conventional active learning and DECAL methods and recorded the earliest round where average accuracy is greater than random chance (33%). Next, the computed the percentage increase/decrease that DECAL achieving relative to the corresponding baseline. FIG. 5D shows the results.

Baseline Sample Acquisition Algorithms. The third study applied patient identifiers as a modular “plug-in” constraint prior to sample selection with each of these baseline algorithms to make our framework clinically deployable. The first baseline employed standard random sampling, the next three are margin, least confidence and entropy uncertainty based sampling (Settles (2009)) and the last was an amalgamation of diversity and uncertainty-based sampling approaches known as BADGE (Ash et al. (2019)).

Results. It can be observed that DECAL consistently matched or surpassed the baseline algorithms.

### Discussion

Active learning aims to find the optimal subset of samples from a dataset for a machine learning model to learn a task well (Dasgupta (2011); Settles (2009)). It is studied because of its ability to reduce the costly and laborious burden on experts to provide data annotations. Typical setups focus on acquisition functions that measure the informativeness of samples using constructs from ensemble learning (Beluch et al. (2018)); probabilistic uncertainty (Gal et al. (2017); Hanneke et al. (2014)) and data representation (Geifman and El-Yaniv (2017); Sener and Savarese (2017)). These works were originally developed for the natural image domain, and although several studies have adapted these and other techniques to medical imagery (Logan et al. (2022); Melendez et al. (2016); Nath et al. (2020); Otalora et al. (2017); Shi et al. (2019)), they have not been adopted or utilized in real clinical settings.

One reason for this non-adoption is that conventional active learning does not follow the diagnostic process. This is because of the experimental settings in natural images that aided the development of existing active learning algorithms (Ash et al. (2019); Hsu and Lin (2015); Sener and Savarese (2017)). Natural images typically contain homogeneous class attributes that can be extracted from the images themselves. Also, these attributes are usually enough to distinguish between classes. However, in medicine, pathologies manifest themselves in visually diverse formats across multiple patients. For example, the characteristics of an aged healthy person are visually different from those of a young healthy person. Doctors overcome this by including clinical data from EMR to assist with their arrival at a diagnostic decision (Brundin-Mather et al. (2018); Brush Jr et al. (2017)). EMR can include patient ID, demographics, diagnostic imaging, and test results that allow a clinician to make a diagnosis.

The exemplary active learning operation of the third study can be designed within a bi-modal interface so as to add practicality to the paradigm for medical image classification. The third study evaluated a classification framework (DECAL) that integrates EMR data. The third study showed that DECAL can aid existing active learning algorithms in finding the best subset for labeling as well as initializing the active learning framework. As such, DECAL is a plug-in approach on top of existing active learning-based methods.

Several works handle multi-modal data by fusing and transforming two heterogeneous modalities into a meaningful format for the model [1-4]. However, these method often add more parameters to increase model complexity which degrade the active learning operations. Existing active learning strategies developed for natural images assume that all the information needed to make a decision can be captured solely from imagery [5]. Therefore, they do not capitalize on additional information present in other modalities. Existing active learning strategy do not use multimodal auxiliary information the way described herein applying it as a constraint to the sample selection process.

### Conclusion

Various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an.” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method. but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1. 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

It should be appreciated that the logical operations described above and, in the appendix, can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The following patents, applications and publications as listed below and throughout this document are hereby incorporated by reference in their entirety herein.

### First Reference Set

[1] Kyle Strimbu and Jorge A Tavel, “What are biomarkers?,” Current Opinion in HIV and AIDS, vol. 5, no. 6, pp. 463, 2010.
[2] Ashish Markan, Aniruddha Agarwal, Atul Arora, Krinjeela Bazgain, Vipin Rana, and Vishali Gupta, “Novel imaging biomarkers in diabetic retinopathy and diabetic macular edema,” Therapeutic Advances in Ophthalmology, vol. 12, pp. 2515841420950513, 2020.
[3] Robert J McDonald, Kara M Schwartz, Laurence J Eckel, Felix E Diehn, Christopher H Hunt, Brian J Bartholmai, Bradley J Erickson, and David F Kallmes, “The effects of changes in utilization and technological advancements of cross-sectional imaging on radiologist workload,” Academic radiology, vol. 22, no. 9, pp. 1191-1198, 2015.
[4] Ghassan AlRegib and Mohit Prabhushankar, “Explanatory paradigms in neural networks,” arXiv preprint arXiv:2202.11838, 2022.
[5] Mark W Johnson, “Posterior vitreous detachment: evolution and complications of its early stages,” American journal of ophthalmology, vol. 149, no. 3, pp. 371-382, 2010.
[6] Amy E Cha, Maria A Villarroel, and Anjel Vahratian, “Eye disorders and vision loss among us adults aged 45 and over with diagnosed diabetes, 2016-2017.” 2019.
[7] Rosana Zacarias Hannouche, Marcos Pereira de A'vila, David Leonardo Cruvinel Isaac, Alan Ricardo Rassi, et al., “Correlation between central subfield thickness, visual acuity and structural changes in diabetic macular edema,” Arquivos brasileiros de oftalmologia, vol. 75, no. 3, pp. 183-187, 2012.
[8] Jennifer K Sun, Michael M Lin, Jan Lammer, Sonja Prager, Rutuparna Sarangi, Paolo S Silva, and Lloyd Paul Aiello, “Disorganization of the retinal inner layers as a predictor of visual acuity in eyes with center involved diabetic macular edema,” JAMA ophthalmology, vol. 132, no. 11, pp. 1309-1316, 2014.
[9] Tomoaki Murakami, Kazuaki Nishijima, Atsushi Sakamoto, Masafumi Ota, Takahiro Horii, and Nagahisa Yoshimura, “Association of pathomorphology, photoreceptor status, and retinal thickness with visual acuity in diabetic retinopathy.” American journal of ophthalmology, vol. 151, no. 2, pp. 310-317, 2011.
[10] Amir H Kashani, Ingrid E Zimmer-Galler, Syed Mahmood Shah, Laurie Dustin, Diana V Do, Dean Eliott, Julia A Haller, and Quan Dong Nguyen, “Retinal thickness analysis by race, gender, and age using stratus oct,” American journal of ophthalmology, vol. 149, no. 3, pp. 496-502, 2010.
[11] Phuc H Le-Khac, Graham Healy, and Alan F Smeaton, “Contrastive representation learning: A framework and review.” IEEE Access, 2020.
[12] Ting Chen, Simon Kornblith, Mohammad Norouzi, and Geoffrey Hinton, “A simple framework for contrastive learning of visual representations,” in International conference on machine learning. PMLR, 2020, pp. 1597-1607.
[13] Prannay Khosla, Piotr Teterwak, Chen Wang, Aaron Sarna, Yonglong Tian, Phillip Isola, Aaron Maschinot, Ce Liu, and Dilip Krishnan, “Supervised contrastive learning.” arXiv preprint arXiv:2004.11362, 2020.
[14] William W Boonn and Curtis P Langlotz, “Radiologist use of and perceived need for patient data access.” Journal of digital imaging, vol. 22, no. 4, pp. 357-362, 2009.
[15] Tao Xu, Han Zhang, Xiaolei Huang, Shaoting Zhang, and Dimitris N Metaxas, “Multimodal deep learning for cervical dysplasia diagnosis,” in International conference on medical image computing and computer assisted intervention. Springer, 2016, pp. 115-123.
[16] Fan Zhang, Zhenzhen Li, Boyan Zhang, Haishun Du, Binjie Wang, and Xinhong Zhang. “Multi-modal deep learning model for auxiliary diagnosis of alzheimer's disease,” Neurocomputing, vol. 361, pp. 185-195, 2019.
[17] P Kharazmi, S Kalia, H Lui, Z J Wang, and T K Lee, “A feature fusion system for basal cell carcinoma detection through data-driven feature learning and patient profile,” Skin research and technology, vol. 24, no. 2, pp. 256-264, 2018.
[18] Jordan Yap, William Yolland, and Philipp Tschandl, “Multimodal skin lesion classification using deep learning,” Experimental dermatology, vol. 27, no. 11, pp. 1261-1267, 2018.
[19] Zina Ben Miled, Kyle Haas, Christopher M Black, Rezaul Karim Khandker, Vasu Chandrasekaran, Richard Lipton, and Malaz A Boustani, “Predicting dementia with routine care emr data,” Artificial intelligence in medicine, vol. 102, pp. 101771, 2020.
[20] B Rajalingam and R Priya, “Multimodal medical image fusion based on deep learning neural network for clinical treatment analysis,” International Journal of ChemTech Research, vol. 11, no. 06, pp. 160-176, 2018.
[21] Siqi Liu, Sidong Liu, Weidong Cai, Hangyu Che, Sonia Pujol, Ron Kikinis, Dagan Feng, Michael J Fulham, et al., “Multimodal neuroimaging feature learning for multiclass diagnosis of alzheimer's disease,” IEEE transactions on biomedical engineering, vol. 62, no. 4, pp. 1132-1140, 2014.
[22] Vaishnavi Subramanian, Minh N Do, and Tanveer Syeda-Mahmood, “Multimodal fusion of imaging and genomics for lung cancer recurrence prediction,” in 2020 IEEE 17th International Symposium on Biomedical Imaging (ISBI). IEEE, 2020, pp. 804-808.
[23] Cecilia S Lee, Doug M Baughman, and Aaron Y Lee, “Deep learning is effective for classifying normal versus age-related macular degeneration oct images,” Ophthalmology Retina, vol. 1, no. 4, pp. 322-327, 2017.
[24] Dogancan Temel, Melvin J Mathew, Ghassan AlRegib, and Yousuf M Khalifa, “Relative afferent pupillary defect screening through transfer learning,” IEEE Journal of Biomedical and Health Informatics, vol. 24, no. 3, pp. 788-795, 2019.
[25] Daniel S Kermany, Michael Goldbaum, Wenjia Cai, Carolina C S Valentim, Huiying Liang, Sally L Baxter, Alex McKeown, Ge Yang, Xiaokang Wu, Fangbing Yan, et al., “Identifying medical diagnoses and treatable diseases by image-based deep learning,” Cell, vol. 172, no. 5, pp. 1122-1131, 2018.
[26] Yash Logan, Kiran Kokilepersaud, Gukyeong Kwon, Ghassan AlRegib, Charles Wykoff, and Hannah Yu, “Multi-modal learning using physicians diagnostics for optical coherence tomography classification.” IEEE International Symposium on Biomedical Imaging (ISBI), 2022.
[27] Mohit Prabhushankar and Ghassan AlRegib, “Contrastive reasoning in neural networks,” arXiv preprint arXiv:2103.12329, 2021.
[28] Thomas Schlegl, Sebastian M Waldstein, Hrvoje Bogunovic, Franz Endstraßer, Amir Sadeghipour, Ana-Maria Philip, Dominika Podkowinski, Bianca S Gerendas, Georg Langs, and Ursula Schmidt-Erfurth, “Fully automated detection and quantification of macular fluid in oct using deep learning.” Ophthalmology, vol. 125, no. 4, pp. 549-558, 2018.
[29] Jeffrey De Fauw, Joseph R Ledsam, Bernardino Romera-Paredes, Stanislav Nikolov, Nenad Tomasev, Sam Blackwell, Harry Askham, Xavier Glorot, Brendan O'Donoghue, Daniel Visentin, et al., “Clinically applicable deep learning for diagnosis and referral in retinal disease,” Nature medicine, vol. 24, no. 9, pp. 1342-1350, 2018.
[30] Mike Pekala, Neil Joshi, T Y Alvin Liu, Neil M Bressler, D Cabrera DeBuc, and Philippe Burlina, “Deep learning based retinal oct segmentation.” Computers in biology and medicine, vol. 114, pp. 103445, 2019.
[31] Michael G Kawczynski, Thomas Bengtsson, Jian Dai, J Jill Hopkins, Simon S Gao, and Jeffrey R Willis, “Development of deep learning models to predict best-corrected visual acuity from optical coherence tomography.” Translational vision science & technology, vol. 9, no. 2, pp. 51-51, 2020.
[32] Filippo Arcadu, Fethallah Benmansour, Andreas Maunz, John Michon, Zdenka Haskova, Dana McClintock, Anthony P Adamis, Jeffrey R Willis, and Marco Prunotto, “Deep learning predicts oct measures of diabetic macular thickening from color fundus photographs.” Investigative ophthalmology & visual science, vol. 60, no. 4, pp. 852-857, 2019.
[33] Kaiming He, Haoqi Fan, Yuxin Wu, Saining Xie, and Ross Girshick, “Momentum contrast for unsupervised visual representation learning.” in Proceedings of the IEEE/CVF Conference on Computer Vision and Pattern Recognition, 2020, pp. 9729-9738.
[34] Mathilde Caron, Ishan Misra, Julien Mairal, Priya Goyal, Piotr Bojanowski, and Armand Joulin, “Unsupervised learning of visual features by contrasting cluster assignments,” arXiv preprint arXiv:2006.09882, 2020.
[35] Jean-Bastien Grill, Florian Strub, Florent Altch'e, Corentin Tallec, Pierre H Richemond, Elena Buchatskaya, Carl Doersch, Bernardo Avila Pires, Zhaohan Daniel Guo, Mohammad Gheshlaghi Azar, et al., “Bootstrap your own latent: A new approach to self-supervised learning,” arXiv preprint arXiv:2006.07733, 2020.
[36] Yazeed Alaudah, Motaz Alfarraj, and Ghassan AlRegib, “Structure label prediction using similarity-based retrieval and weakly supervised label mapping structure label prediction,” Geophysics, vol. 84, no. 1, pp. V67- V79, 2019.
[37] Yazeed Alaudah, Shan Gao, and Ghassan AlRegib, “Learning to label seismic structures with deconvolution networks and weak labels,” in 2018 SEG International Exposition and Annual Meeting. OnePetro, 2018.
[38] Kiran Kokilepersaud, Mohit Prabhushankar, and Ghassan AlRegib, “Volumetric supervised contrastive learning for seismic semantic segmentation,” arXiv preprint arXiv:2206.08158, 2022.
[39] Antoine Rivail, Ursula Schmidt-Erfurth, Wolf-Dieter Vogl, Sebastian M Waldstein, Sophie Riedl, Christoph Grechenig, Zhichao Wu, and Hrvoje Bogunovic, “Modeling disease progression in retinal octs with longitudinal self-supervised learning,” in International Workshop on Predictive Intelligence In MEdicine. Springer, 2019, pp. 44-52.
[40] Yuhan Zhang, Mingchao Li, Zexuan Ji, Wen Fan, Songtao Yuan, Qinghuai Liu, and Qiang Chen, “Twin self-supervision based semisupervised learning (ts-ssl): Retinal anomaly classification in sd-oct images,” Neurocomputing, 2021.
[41] Jiaming Qiu and Yankui Sun, “Self-supervised iterative refinement learning for macular oct volumetric data classification,” Computers in biology and medicine, vol. 111, pp. 103327, 2019.
[42] Jia Deng, Wei Dong, Richard Socher, Li-Jia Li, Kai Li, and Li Fei-Fei, “Imagenet: A large-scale hierarchical image database,” in 2009 IEEE conference on computer vision and pattern recognition. Ieee, 2009, pp. 248-255.
[43] Hari Sowrirajan, Jingbo Yang, Andrew Y Ng, and Pranav Rajpurkar, “Moco-cxr: Moco pretraining improves representation and transferability of chest x-ray models,” arXiv preprint arXiv:2010.05352, 2020.
[44] Shekoofeh Azizi, Basil Mustafa, Fiona Ryan, Zachary Beaver, Jan Freyberg, Jonathan Deaton, Aaron Loh, Alan Karthikesalingam, Simon Kornblith, Ting Chen, et al., “Big self-supervised models advance medical image classification,” arXiv preprint arXiv:2101.05224, 2021.
[45] Yen-Pin Chen, Yuan-Hsun Lo, Feipei Lai, and Chien-Hua Huang, “Disease concept-embedding based on the self-supervised method for medical information extraction from electronic health records and disease retrieval: Algorithm development and validation study,” Journal of Medical Internet Research, vol. 23, no. 1, pp. e25113, 2021.
[46] Yuhao Zhang, Hang Jiang, Yasuhide Miura, Christopher D Manning, and Curtis P Langlotz, “Contrastive learning of medical visual representations from paired images and text,” arXiv preprint arXiv:2010.00747, 2020.
[47] Yen Nhi Truong Vu, Richard Wang, Niranjan Balachandar, Can Liu, Andrew Y Ng, and Pranav Rajpurkar, “Medaug: Contrastive learning leveraging patient metadata improves representations for chest x-ray interpretation,” arXiv preprint arXiv:2102.10663, 2021.
[48] Gongbo Liang, Connor Greenwell, Yu Zhang, Xin Xing, Xiaoqin Wang, Ramakanth Kavuluru, and Nathan Jacobs, “Contrastive cross-modal pretraining: A general strategy for small sample medical imaging,” IEEE Journal of Biomedical and Health Informatics, vol. 26, no. 4, pp. 1640-1649, 2021.
[49] Zhao Wang, Quande Liu, and Qi Dou, “Contrastive cross-site learning with redesigned net for covid-19 ct classification,” IEEE Journal of Biomedical and Health Informatics, vol. 24, no. 10, pp. 2806-2813, 2020.
[50] Daniel Kermany, Kang Zhang, Michael Goldbaum, et al., “Labeled optical coherence tomography (oct) and chest x-ray images for classification,” Mendeley data, vol. 2, no. 2,2018.
[51] Sina Farsiu, Stephanie J Chiu, Rachelle V O'Connell, Francisco A Folgar, Eric Yuan, Joseph A Izatt, Cynthia A Toth, Age-Related Eye Disease Study 2 Ancillary Spectral Domain Optical Coherence Tomography Study Group, et al., “Quantitative classification of eyes with and without intermediate age-related macular degeneration using optical coherence tomography.” Ophthalmology, vol. 121, no. 1, pp. 162-172, 2014.
[52] Martina Melin\check{ }s\check{ }cak, Marin Radmilovi'c, Zoran Vatavuk, and Sven Lon\check{ }cari'c, “Annotated retinal optical coherence tomography images (aroi) database for joint retinal layer and fluid segmentation,” Automatika: \check{ }casopis za automatiku, mjerenje, elektroniku, ra\check{ }cunarstvo I komunikacije, vol. 62, no. 3-4, pp. 375-385, 2021.
[53] Stephanie J Chiu, Michael J Allingham, Priyatham S Mettu, Scott W Cousins, Joseph A Izatt, and Sina Farsiu, “Kernel regression based segmentation of optical coherence tomography images with diabetic macular edema.” Biomedical optics express, vol. 6. no. 4, pp. 1172-1194, 2015.
[54] J Yu Hannah, Justis P Ehlers, Duriye Damla Sevgi, Jenna Hach, Margaret O'Connell, Jamie L Reese, Sunil K Srivastava, and Charles C Wykoff, “Real-time photographic- and fluorescein angiographic-guided management of diabetic retinopathy: Randomized prime trial outcomes,” American Journal of Ophthalmology, vol. 226, pp. 126-136, 2021.
[55] John F Payne, Charles C Wykoff, W Lloyd Clark, Beau B Bruce, David S Boyer, David M Brown, TREX-DME study group, et al., “Randomized trial of treat and extend ranibizumab with and without navigated laser for diabetic macular edema: Trex-dme 1 year outcomes,” Ophthalmology, vol. 124, no. 1, pp. 74-81, 2017.
[56] John F Payne, Charles C Wykoff, W Lloyd Clark, Beau B Bruce, David S Boyer, David M Brown, John A Wells III, David L Johnson, Matthew Benz, Eric Chen, et al., “Randomized trial of treat and extend ranibizumab with and without navigated laser versus monthly dosing for diabetic macular edema: Trex-dme 2-year outcomes,” American journal of ophthalmology, vol. 202, pp. 91-99, 2019.
[57] John F Payne, Charles C Wykoff, W Lloyd Clark, Beau B Bruce, David S Boyer, and David M Brown, “Long-term outcomes of treat-and-extend ranibizumab with and without navigated laser for diabetic macular oedema: Trex-dme 3-year results,” British Journal of Ophthalmology, vol. 105, no. 2, pp. 253-257, 2021.
[58] Charles C Wykoff, Muneeswar G Nittala, Brenda Zhou, Wenying Fan, Swetha Bindu Velaga, Shaun I R Lampen, Alexander M Rusakevich, Justis P Ehlers, Amy Babiuch, David M Brown, et al., “Intravitreal aflibercept for retinal nonperfusion in proliferative diabetic retinopathy: outcomes from the randomized recovery trial,” Ophthalmology Retina, vol. 3, no. 12, pp. 1076-1086, 2019.
[59] Mohit Prabhushankar, Kiran Kokilepersaud, Yash-yee Logan, Stephanie Trejo Corona, Ghassan AlRegib, and Charles Wykoff, “Olives dataset: Ophthalmic labels for investigating visual eye semantics,” in Proceedings of the Neural Information Processing Systems Track on Datasets and Benchmarks 2 (NeurIPS Datasets and Benchmarks 2022), Under Review.
[60] Kaiming He, Xiangyu Zhang, Shaoqing Ren, and Jian Sun, “Deep residual learning for image recognition,” in Proceedings of the IEEE conference on computer vision and pattern recognition, 2016, pp. 770-778.
[61] Laurens Van der Maaten and Geoffrey Hinton, “Visualizing data using t-sne.,” Journal of machine learning research, vol. 9, no. 11, 2008.
[62] Junnan Li, Pan Zhou, Caiming Xiong, and Steven C H Hoi, “Prototypical contrastive learning of unsupervised representations,” arXiv preprint arXiv:2005.04966, 2020.
[63] Xinlei Chen, Haoqi Fan, Ross Girshick, and Kaiming He, “Improved baselines with momentum contrastive learning.” arXiv preprint arXiv:2003.04297, 2020.
[64] Elijah Cole, Xuan Yang, Kimberly Wilber, Oisin Mac Aodha, and Serge Belongie, “When does contrastive visual representation learning work?,” arXiv preprint arXiv:2105.05837, 2021.
[65] Mohit Prabhushankar, Gukyeong Kwon, Dogancan Temel, and Ghassan AlRegib, “Contrastive explanations in neural networks,” in 2020 IEEE International Conference on Image Processing (ICIP). IEEE, 2020, pp. 3289-3293.
[66] Sanjeev Arora, Hrishikesh Khandeparkar, Mikhail Khodak, Orestis Plevrakis, and Nikunj Saunshi, “A theoretical analysis of contrastive unsupervised representation learning.” arXiv preprint arXiv: 1902.09229, 2019.

### Second Reference Set

[1A] Chen, T., Kornblith, S., Norouzi, M., & Hinton, G. (2020, November). A simple framework for contrastive learning of visual representations. In International conference on machine learning (pp. 1597-1607). PMLR.
[2A] Chen, X., Fan, H., Girshick, R., & He, K. (2020). Improved baselines with momentum contrastive learning. arXiv preprint arXiv:2003.04297.
[3A] Li, J., Zhou, P., Xiong, C., & Hoi, S. C. (2020). Prototypical contrastive learning of unsupervised representations. arXiv preprint arXiv: 2005.04966.
[4A] Azizi, S., Mustafa, B., Ryan, F., Beaver, Z., Freyberg, J., Deaton, J., . . . & Norouzi, M. (2021). Big self-supervised models advance medical image classification. In Proceedings of the IEEE/CVF International Conference on Computer Vision (pp. 3478-3488).
[5A] Chen, Y. P., Lo, Y. H., Lai, F., & Huang, C. H. (2021). Disease concept-embedding based on the self-supervised method for medical information extraction from electronic health records and disease retrieval: Algorithm development and validation study. Journal of Medical Internet Research, 23(1), e25113.
[6A] Vu, Y. N. T., Wang, R., Balachandar, N., Liu, C., Ng, A. Y., & Rajpurkar, P. (2021, October). Medaug: Contrastive learning leveraging patient metadata improves representations for chest x-ray interpretation. In Machine Learning for Healthcare Conference (pp. 755-769). PMLR.

### Third Reference Set

[1B] A. Krizhevsky, I. Sutskever, and G. E. Hinton, “Imagenet classification with deep convolutional neural networks,” Advances in neural information processing systems, vol. 25, pp. 1097-1105, 2012.
[2B] T. Mikolov, I. Sutskever, K. Chen, G. S. Corrado, and J. Dean, “Distributed representation of words and phrases and their compositionality,” in Advances in neural information processing systems, 2013, pp. 3111-3119.
[3B] T. BaltruË‡saitis, C. Ahuja, and L.-P. Morency, “Multimodal machine learning: A survey and taxonomy,” IEEE transactions on pattern analysis and machine intelligence, vol. 41, no. 2, pp. 423-443, 2018.
[4B] S.-C. Huang, A. Pareek, S. Seyyedi, I. Banerjee, and M. P. Lungren, “Fusion of medical imaging and electronic health records using deep learning: A systematic review and implementation guidelines,” NPJ digital medicine, vol. 3, no. 1, pp. 1-9, 2020
[5B] Budd, Samuel, Emma C. Robinson, and Bernhard Kainz. “A survey on active learning and human-in-the-loop deep learning for medical image analysis.” Medical Image Analysis 71 (2021): 102062.
[6B] Jordan T Ash, Chicheng Zhang, Akshay Krishnamurthy, John Langford, and Alekh Agarwal. Deep batch active learning by diverse, uncertain gradient lower bounds. arXiv preprint arXiv:1906.03671, 2019.
[7B] William H Beluch, Tim Genewein, Andreas Nurnberger, and Jan M Kohler. The power of ensembles for active learning in image classification. In Proceedings of the IEEE conference on computer vision and pattern recognition, pages 9368{9377, 2018.
[8B] Rebecca Brundin-Mather, Andrea Soo, Danny J Zuege, Daniel J Niven, Kirsten Fiest, Christopher J Doig, David Zygun, Jamie M Boyd, Jeanna Parsons Leigh, Sean M Bagshaw, et al. Secondary emr data for quality improvement and research: a comparison of manual and electronic data collection from an integrated critical care electronic medical record system. Journal of critical care, 47:295{301, 2018.
[9B] John E Brush Jr, Jonathan Sherbino, and Geoffrey R Norman. How expert clinicians intuitively recognize a medical diagnosis. The American journal of medicine, 130(6): 629{634, 2017.
[10B] Sanjoy Dasgupta. Two faces of active learning. Theoretical computer science, 412(19): 1767{1781, 2011.
[11B] Yarin Gal, Riashat Islam, and Zoubin Ghahramani. Deep bayesian active learning with image data. In International Conference on Machine Learning, pages 1183{1192. PMLR, 2017.
[12B] Yonatan Geifman and Ran El-Yaniv. Deep active learning over the long tail. arXiv preprint arXiv:1711.00941, 2017.
[13B] Steve Hanneke et al. Theory of disagreement-based active learning. Foundations and Trends® in Machine Learning, 7(2-3):131{309, 2014.
[14B] Kaiming He, Xiangyu Zhang, Shaoqing Ren, and Jian Sun. Deep residual learning for image recognition. In Proceedings of the IEEE conference on computer vision and pattern recognition, pages 770{778, 2016.
[15B] Wei-Ning Hsu and Hsuan-Tien Lin. Active learning by learning. In Twenty-Ninth AAAI conference on artificial intelligence, 2015.
[16B] Gao Huang, Zhuang Liu, Laurens Van Der Maaten, and Kilian Q Weinberger. Densely connected convolutional networks. In Proceedings of the IEEE conference on computer vision and pattern recognition, pages 4700{4708, 2017.
[17B] Daniel S Kermany, Michael Goldbaum, Wenjia Cai, Carolina C S Valentim, Huiying Liang, Sally L Baxter, Alex McKeown, Ge Yang, Xiaokang Wu, Fangbing Yan, et al. Identifying medical diagnoses and treatable diseases by image-based deep learning. Cell, 172(5): 1122{1131, 2018.
[18B] Kiran Kokilepersaud, Mohit Prabhushankar, Ghassan AlRegib, Stephanie Trejo Corona, and Charles Wykoff. Gradient-based severity labeling for biomarker classification in oct. In International Conference on Image Processing (ICIP). IEEE, 2022.
[19B] Yash-yee Logan, Ryan Benkert, Ahmad Mustafa, and Ghassan AlRegib. Patient aware active learning for fine-grained oct classification. In International Conference on Image Processing (ICIP). IEEE, 2022.
[20B] Yash-yee Logan*, Kiran Kokilepersaud*, Stephanie Trejo Corona, Mohit Prabhushankar, Ghassan AlRegib, and Charles Wykoff. Olives: Optical labels for investigating visual eye semantics. In Neural Information Processing Systems (NeurIPS) Track on Datasets and Benchmarks. IEEE, 2022 Under Review.
[21B] Jaime Melendez, Bram van Ginneken, Pragnya Maduskar, Rick H. H. M. Philipsen, Helen Ayles, and Clara I. Sanchez. On combining multiple-instance learning and active learning for computer-aided detection of tuberculosis. IEEE Transactions on Medical Imaging. 35 (4):1013{1024, 2016. doi: 10.1109/TMI.2015.2505672.
[22B] Vishwesh Nath, Dong Yang, Bennett A Landman, Daguang Xu, and Holger R Roth. Diminishing uncertainty within the training pool: Active learning for medical image segmentation. IEEE Transactions on Medical Imaging, 40(10):2534{2547, 2020.
[23B] Sebastian Otalora, Oscar Perdomo, Fabio Gonzalez, and Henning Muller. Training deep convolutional neural networks with active learning for exudate classification in eye fundus images. In Intravascular imaging and computer assisted stenting, and large-scale annotation of biomedical data and expert label synthesis, pages 146{154. Springer, 2017.
[24B] Ozan Sener and Silvio Savarese. Active learning for convolutional neural networks: A core-set approach. arXiv preprint arXiv:1708.00489, 2017.
[25B] Burr Settles. Active learning literature survey. 2009.
[26B] Xueying Shi, Qi Dou, Cheng Xue, Jing Qin, Hao Chen, and Pheng-Ann Heng. An active learning approach for reducing annotation cost in skin lesion analysis. In International Workshop on Machine Learning in Medical Imaging, pages 628{636. Springer, 2019.
[27B] Jingbo Zhu, Huizhen Wang, Tianshun Yao, and Benjamin K Tsou. Active learning with sampling by uncertainty and density for word sense disambiguation and text classification. In Proceedings of the 22nd International Conference on Computational Linguistics (Coling 2008), pages 1137{1144, 2008.

What is claimed is:

1. A method for asymmetric training an AI model, the method comprising:
receiving a multi-modal dataset including a first image data set and a second image data set, wherein the first image data set includes a metadata label as a medical condition in an electronic medical record of a patient, and wherein the second image data set includes clinical labels;
performing training of an AI model using the first dataset using the metadata labels to adjust first weights in the AI model; and
performing contrastive learning of the AI model using the second dataset, wherein the AI model includes a first portion having the first weights and a second portion having second weights, wherein the contrastive learning held constant the first weights of the AI model and adjusted the second weights of the AI model via a contrastive loss function using the clinical labels, and wherein the second dataset has a value of a presence of the medical condition in the metadata label.

2. The method of claim 1, wherein the step of performing the supervised learning of an AI model includes:
providing a clinically labeled augmented batch having the metadata label;
forward propagating through the AI model;
varying a projection network coupled to the AI model; and
computing a loss function at the output of the projection network to adjust the AI model.

3. The method of claim 1, further comprising:
outputting, via a report or display, classifier output of the second AI model, wherein the classifier output is used for diagnosis of a disease or a medical condition.

4. The method of claim 1, wherein the first data set comprises image data from a medical scan.

5. The method of claim 1, wherein the first data set comprises image data from a sensor.

6. The method of claim 1, wherein the first portion of the AI model comprises an autoencoder.

7. The method of claim 1, wherein the second portion of the AI model comprises a linear layer appended to the first portion.

8. The method of claim 1, wherein the second portion of the AI model comprises a semantic segmentation head appended to the first portion.

9. The method of claim 7, wherein the biomarker data includes at least one of:
Intraretinal Fluid (IRF), Diabetic Macular Edema (DME), and Intra-Retinal Hyper-Reflective Foci (IRHRF).

10. The method of claim 1, wherein the training operation is configured to:
compute a distribution of unique identifier for subjects throughout an unlabeled data set; and
sample for the training operation based on the computed distribution.

11. A system comprising:
a processor; and
a memory having instructions stored thereon, wherein execution of the instructions by the processor causes the processor to:
receive a multi-modal dataset including a first image data set and a second image data set, wherein the first image data set includes a metadata label as a medical condition in an electronic medical record of a patient, and wherein the second image data set includes clinical labels;
perform training of an AI model using the first dataset using the metadata labels to adjust first weights in the AI model; and
perform contrastive learning of the AI model using the second dataset, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the meta data label.

12. The system of claim 11, wherein the instructions to perform the supervised learning of an AI model includes:
instructions to provide a clinically labeled augmented batch having the meta data label;
instructions to forward propagating through the AI model;
instructions to vary a projection network coupled to the AI model; and
instructions to compute a loss function at the output of the projection network to adjust the AI model.

13. The system of claim 11, further comprising:
a sensor, wherein the first data set comprises image data acquired from the sensor.

14. The system of claim 11, wherein the first portion of the AI model comprises an autoencoder.

15. The system of claim 11, wherein the second portion of the AI model comprises at least one of (i) a linear layer appended to the first portion or (ii) a semantic segmentation head appended to the first portion.

16. The system of claim 11, wherein the instructions for the training operation includes:
instructions to compute a distribution of unique identifier for subject throughout an unlabeled pool; and
instructions to sample for the training operation based on the computed distribution.

17. A non-transitory computer readable medium having instructions stored thereon, wherein execution of the instructions by a processor causes the processor to:
receive a multi-modal dataset including a first image data set and a second image data set, wherein the first image data set includes a metadata label as a medical condition in an electronic medical record of a patient, and wherein the second image data set includes clinical labels;
perform training of an AI model using the first dataset using the metadata labels to adjust first weights in the AI model; and
perform contrastive learning of the AI model using the second dataset, wherein the AI model includes a first portion having the first weights and a second portion having second weights, wherein the contrastive learning held constant the first weights of the AI model and adjust the second weights of the AI model via a contrastive loss function using the clinical labels, and wherein the second dataset has a value of a presence of the medical condition in the metadata label.

18. The non-transitory computer-readable medium of claim 17, wherein the instructions to perform the supervised learning of an AI model include:
instructions to provide a clinically labeled augmented batch having the metadata label;
instructions to forward propagating through the AI model;
instructions to vary a projection network coupled to the AI model; and
instructions to compute a loss function at the output of the projection network to adjust the AI model.

19. The non-transitory computer-readable medium of claim 17, wherein the second portion of the AI model comprises at least one of (i) a linear layer appended to the first portion or (ii) a semantic segmentation head appended to the first portion.

20. The non-transitory computer-readable medium of claim 17, wherein the instructions for the training operation include:
instructions to compute a distribution of unique identifiers for a subject throughout an unlabeled data set; and
instructions to sample for the training operation based on the computed distribution.
